(19) United States
(12) Patent Application Publication
(10) Pub. No.: US 2016/0282367 A1
(43) Pub. Date: Sep. 29, 2016

(54) METHODS FOR CALIBRATED ION MOBILITY ANALYSIS AND USES THEREOF

(71) Applicant: UNIVERSITY OF WASHINGTON THROUGH ITS CENTER FOR CO MMERCIALIZATION, Seattle, WA (US)

(72) Inventors: Jay HEINECKE, Seattle, WA (US); Patrick Moore HUTCHINS, Seattle, WA (US)

(73) Assignee: UNIVERSITY OF WASHINGTON THROUGH ITS CENTER FOR CO MMERCIALIZATION, Seattle, WA (US)

(21) Appl. No.: 15/036,901

(22) PCT Filed: Nov. 25, 2014
(86) PCT No.: PCT/US14/67419

§ 371 Date: May 16, 2016

Related U.S. Application Data
(60) Provisional application No. 61/908,623, filed on Nov. 25, 2013.
(60) Provisional application No. 62/054,233, filed on Sep. 23, 2014.

(51) Int. Cl. G01N 33/92; C12Q 1/26

(52) U.S. Cl. CPC G01N 33/92; C12Q 1/26; G01N 2333/775

(57) ABSTRACT

The present invention relates to improved ion mobility analysis (IMA) methods that can accurately quantify particle concentration in a sample solution. Specifically, reference particles of known solution-phase concentration are used for calibration. In addition, by exploiting spectral deconvolution techniques, the concentrations of subpopulations within the particles can also be accurately quantified. The improved IMA methods permit, for the first time, the quantification of absolute concentrations of HDL particles and subpopulations thereof in a biological sample. The correlations of HDL particle concentrations and conditions such as LCAT deficiency and cardiovascular diseases have been established. Accordingly, the present invention also provide methods to determine whether a subject is at risk to develop or is suffering from these conditions by using HDL particle concentration as a clinical metric.

### CROSS-REFERENCE TO RELATED APPLICATION

This application claims benefit under 35 U.S.C. §119(e) of U.S. Provisional Application No. 61/908,623 filed Nov. 25, 2013 and No. 62/054,233 filed Sep. 23, 2014, the contents of each of which are incorporated herein by reference in their entirety.

### GOVERNMENT SUPPORT

This invention was made with government support under grant R01 HL112625 and R01 HL108897 awarded by the National Institutes of Health (NIH). The government has certain rights in the invention.

### TECHNICAL FIELD

The present disclosure relates generally to ion mobility analysis, measurement of HDL particle concentration, and cardiovascular disease (CVD) risk assessment.

### BACKGROUND

It is important to develop new metrics to determine whether HDL is cardioprotective in humans. Plasma concentrations of HDL cholesterol (HDL-C) are widely used clinically to assess HDL's cardioprotective potential. There is a robust, inverse association of HDL-C with cardiovascular disease (CVD) risk in clinical, epidemiological, and genetic studies. However, recent work has cast doubt on the hypothesis that the concentration of HDL-C captures its proposed cardioprotective functions. For example, genetic variations that alter concentrations of HDL-C do not always predict CVD risk. Strikingly, a cholesteryl ester transfer protein inhibitor and niacin, two interventions that increase HDL-C, failed to reduce CVD risk in statin-treated humans with established CVD. These observations indicate that HDL-C concentrations do not always predict CVD risk and that increasing HDL-C is not necessarily therapeutic.

It is important to note that many lines of evidence strongly suggest that HDL directly protects against vascular disease. For example, a polymorphism in apolipoprotein A-I (apoA-I), the major HDL protein, associates with low HDL cholesterol concentrations and premature coronary artery disease. Also, humans with familial deficiency of apoA-I, the major HDL protein, suffer severe early-onset CVD. Furthermore, people with Tangier disease [who lack ATP-binding cassette transporter 1 (ABCA1), an important first step in cholesterol export from cells] have very low HDL-C concentrations and accumulate cholesterol-laden macrophages in many different tissues.

These discrepancies highlight a central question: Does HDL deficiency promote human atherosclerosis, or is it simply a marker for other risk factors such as insulin resistance? To make this determination, it is critical to identify HDL metrics that truly reflect CVD risk.

One promising approach is measurement of HDL particle concentration (HDL-P), which characterizes the size and concentration of HDL in plasma. HDL is a collection of macromolecular particles that contain >80 different proteins (Vaisar T, et al., J Clin Invest 2007; 117:746-56; Shah A S, et al., J Lipid Res 2013; 54:2575-85) and range in size from <7 nm to >14 nm (Rosenson R S, et al., Clin Chem 2011; 57:392-410). It is therefore plausible that the plasma concentration of HDL particles (HDL-P)—or of a subset of particles—might better reflect HDL-mediated cardioprotection than surrogate measures of HDL such as cholesterol or apoA-I (Rosenson R S, et al., Clin Chem 2011; 57:392-410; Jeyarajah E J, et al., Clin Lab Med 2006; 26:847-70; Caulfield M P, et al., Clin Chem 2008; 54:1307-16; Mackey R H, et al., J Am Coll Cardiol 2012; 60:508-16; Mora S, et al., Circulation 2013; 128:1189-97; Asztalos B F, et al., Curr Opin Lipidol 2011; 22:176-85; Asztalos B F, Schaefer E J., Am J Cardiol 2003; 91:12-7).

Two methods have been described for quantifying HDL-P in human plasma, one on the basis of nuclear magnetic resonance (NMR) (Jeyarajah E J, et al., Clin Lab Med 2006; 26:847-70; Otvos J D, et al., Clin Chem 1991; 37:377-86), and the other, ion mobility analysis (IMA) (Caulfield M P, et al., Clin Chem 2008; 54:1307-16). To quantify lipoproteins by NMR, the amplitudes of spectral signals emitted by lipoprotein subclasses of different sizes are measured. The data are then reduced with a proprietary algorithm. To quantify HDL by IMA, solvated lipoproteins are introduced into the gas phase by electrospray ionization (ESI). Charged HDL particles are then separated on the basis of their differential mobility through a buffer gas. Although both approaches have helped establish HDL-P as a potentially relevant clinical metric, only limited evidence suggests that it is substantially independent of HDL-C (Mackey R H, et al., J Am Coll Cardiol 2012; 60:508-16; Mora S, et al., Circulation 2013; 128:1189-97). Moreover, the 2 methods give very different mean HDL-P values (approximately 5 mol/L and approximately 30 mol/L), and neither yields a value consistent with the stoichiometry of 3-4 apoA-I/HDL and with the current understanding of HDL structure (Shen B W, et al., Proc Natl Acad Sci USA 1977; 74:837-41; Huang R, et al., Nat Struct Mol Biol 2011; 18:416-22). For example, 7 independent studies using existing IMA methods indicate a mean stoichiometry of almost 10 apoA-I molecules per HDL particle (see Table 4). In contrast, NMR analyses indicate a stoichiometry of approximately 1.6 apoA-I molecules per HDL particle (see Table 4).

To determine whether HDL-P can be a valid clinical metric, it will be important to resolve these discrepancies. And accordingly, there is a need in the art for new methods to accurately quantify the concentration of HDL-P in a blood sample.

### SUMMARY

Among other things, the technology described herein provides improved IMA methods that can accurately quantify the concentration of HDL-P in a blood sample. For example, the improved IMA methods provided herein led to the determination of about 3.6 apoA-I/HDL, in excellent agreement with the current understanding of HDL structure.

Ion mobility can accurately measure the concentration of particles in the gas phase because it rests on well-established physical principles. However, for particles in a solution, many factors affect the production of gas-phase ions from the solution during ionization such as electrospray ionization, an important step of IMA. Because the generation and transmission of ions by ionization is variable, quantitative assays of aqueous particles on the basis of this approach must account for ionization efficiency and other sources of signal loss.

The technology described herein is based, in part, on the surprising discovery that ionization efficiency and other sources of signal loss can be accounted for by a calibration step, where IMA is performed on particles of known solution-phase concentration. It has been surprisingly discovered, among other things, that different particles in solutions—even those having different diameters, material properties, or physiochemical properties—elicit similar responses when analyzed by the same IMA instrument (see FIGS. 1B & 1C). This discovery thus permits the use of particles of known solution-phase concentration to calibrate the IMA system for quantitative measurements of particles in the solution phase.

Furthermore, it has been discovered that a spectrum obtained from IMA can be processed via adaptive peak fitting to identify subspecies within a population of particles. For example, five subspecies or subpopulations of HDL-P have been identified using calibrated IMA. The identification of these subspecies and the quantification thereof permit a skilled artisan to correlate them with a variety of conditions such as cardiovascular diseases, which was not possible previously.

Accordingly, one aspect of the technology described herein relates to a method of characterizing particles in a sample solution, the method comprising: (i) converting a portion of the particles in the sample solution into gas-phase ions; (ii) performing an ion mobility measurement on the gas-phase ions, whereby the gas-phase ions are enumerated according to size, thereby producing data relating particle size to relative abundance; (iii) processing the data by using a calibration regression, wherein the calibration regression is obtained by: (a) performing steps (i) and (ii) on reference particles of known solution-phase concentration; and (b) constructing the regression relating total number of enumerated gas-phase ions of the reference particles to the known solution-phase concentration; and (iv) quantitatively determining particle concentration in the sample solution based on the processing.

In one embodiment, step (ii) of the method produces a spectrum of particle size distribution.

In one embodiment, the method further comprises superimposing a plurality of distribution curves over the spectrum, each distribution curve representing a subpopulation of the gas-phase ions according to size, and iteratively adjusting parameters of the distribution curves to minimize the difference between the spectrum and sum of the distribution curves.

In one embodiment, the distribution curve is selected from the group consisting of a Gaussian, a split Gaussian, a Voigt, a split Voigt, a Pearson7, a split Pearson7, a Lorentzian, and a split Lorentzian distribution.

In one embodiment, the ion mobility measurement comprises introducing the gas-phase ions into an electromagnetic field having an effect on the translation of the ions, thereby inducing an electrophoretic motion.

In one embodiment, the conversion into gas-phase ions is done by electrospray ionization.

In one embodiment, the particles and reference particles are each independently selected from the group consisting of biological particles, inorganic particles, metallic particles, metallo-organic particles, organic particles, polymeric particles, and a combination thereof.

In one embodiment, the biological particles are biological cells, proteins or aggregates thereof, or lipoproteins.

In one embodiment, the lipoproteins are selected from the group consisting of whole HDL, fractionated HDL, whole LDL, fractionated LDL, whole VLDL, fractionated VLDL, and a combination thereof.

In one embodiment, the reference particles comprises nanoparticles selected from the group consisting of gold, silver, polystyrene, silica, purified proteins, and a combination thereof.

In one embodiment, the purified protein is glucose oxidase.

In one embodiment, the sample solution is an aqueous solution.

In one embodiment, the aqueous solution is a biological sample.

In one embodiment, the biological sample is selected from the group consisting of blood, plasma, serum, urine, cerebrospinal fluid, and saliva.

In one embodiment, the method further comprises dialyzing the aqueous solution to substantially remove salts.

In one embodiment, the reference particles are of known molecular weight.

In one embodiment, method further comprises determining the molecular weight of the particles being characterized.

In one embodiment, the reference particles are of known size.

Another aspect of the technology described herein relates to a method of determining if a subject is at risk to develop or is suffering from a cardiovascular disease, the method comprising: measuring, in a biological sample obtained from the subject, the size and concentration of HDL particles according to the calibrated IMA methods described herein.

In one embodiment, the HDL particles are selected from the group consisting of very small HDL particles, small HDL particles, medium HDL particles, large HDL particles, very large HDL particles, and a combination thereof.

In one embodiment, the method further comprises measuring lipoproteins other than HDL.

In one embodiment, the cardiovascular disease is selected from the group consisting of atherosclerosis, coronary vascular disease, ischemic heart disease, myocardial infarction, angina pectoris, peripheral vascular disease, cerebrovascular disease, endothelial dysfunction, and stroke.

In one embodiment, the biological sample is selected from the group consisting of blood, plasma, and serum.

In one embodiment, the subject is a mammal.

In one embodiment, the mammal is a human.

Another aspect of the technology described herein relates to a method of determining if a subject has lecithin-cholesterol acyltransferase deficiency (LCAT), the method comprising: (i) measuring, in a biological sample obtained from the subject, the concentration of HDL particles; and (ii) determining that the subject has LCAT if the concentration of very small HDL particles is at or above a first reference level, and the concentration of at least one other subpopulation of HDL particles is below a second reference level.

In one embodiment, the method further comprises measuring the size of HDL particles.

In one embodiment, the size and concentration of HDL particles are measured according to the calibrated IMA methods described herein.

In one embodiment, the at least one other subpopulation of HDL particles is selected from the group consisting of small HDL particles, medium HDL particles, large HDL particles, very large HDL particles, and a combination thereof.

In one embodiment, when the concentration of very small HDL particles is at or above the first reference level and the concentration of at least one other subpopulation of HDL particles is below a second reference level, the method further comprises administering a treatment appropriate for treating LCAT.

In one embodiment, the method further comprises measuring lipoproteins other than HDL.

In one embodiment, the biological sample is selected from the group consisting of blood, plasma, and serum.

In one embodiment, the subject is a mammal.

In one embodiment, the mammal is a human.

In one embodiment, the first reference level is a concentration of very small HDL particles in a population of healthy subjects.

In one embodiment, the second reference level is a concentration of at least one other subpopulation of HDL particles in a population of healthy subjects.

Another aspect of the technology described herein relates to a method of determining if a subject is at risk to develop or is suffering from atherosclerosis, the method comprising: (i) measuring, in a biological sample obtained from the subject, the concentration of HDL particles; and (ii) determining that the subject is at risk to develop or is suffering from atherosclerosis if the concentration of HDL particles is below a reference level.

In one embodiment, the method further comprises measuring the size of HDL particles.

In one embodiment, the atherosclerosis is selected from the group consisting of coronary artery disease (CAD), carotid cerebrovascular disease (CCVD), and peripheral vascular disease.

In one embodiment, the size and concentration of HDL particles are measured according to the calibrated IMA methods described herein.

In one embodiment, the HDL particles are very small HDL particles.

In one embodiment, the HDL particles are medium HDL particles.

In one embodiment, the HDL particles are total HDL particles.

In one embodiment, when the concentration of HDL particles is below the reference level, the method further comprises administering a treatment appropriate for treating atherosclerosis.

In one embodiment, the reference level is a concentration of HDL particles in a population of healthy subjects.

In one embodiment, the method further comprises measuring lipoproteins other than HDL.

In one embodiment, the biological sample is selected from the group consisting of blood, plasma, and serum.

In one embodiment, the subject is a mammal.

In one embodiment, the mammal is a human.

Yet another aspect of the technology described herein relates to a method of determining if a subject is at risk to develop or is suffering from endothelial dysfunction, the method comprising: (i) measuring, in a biological sample obtained from the subject, the concentration of HDL particles; and (ii) determining that the subject is at risk to develop or is suffering from endothelial dysfunction if the concentration of HDL particles is below a reference level.

In one embodiment, the method further comprises measuring the size of HDL particles.

In one embodiment, the HDL particles are medium HDL particles.

In one embodiment, the size and concentration of HDL particles are measured according to the calibrated IMA methods described herein.

In one embodiment, when the concentration of medium HDL particles is below the reference level, the method further comprises administering a treatment appropriate for treating endothelial dysfunction.

In one embodiment, the method further comprises measuring lipoproteins other than HDL.

In one embodiment, the biological sample is selected from the group consisting of blood, plasma, and serum.

In one embodiment, the subject is a mammal.

In one embodiment, the mammal is a human.

In one embodiment, the reference level is a concentration of HDL particles in a population of healthy subjects.

### DEFINITIONS

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are useful to an embodiment, yet open to the inclusion of unspecified elements, whether useful or not.

As used herein the term “consisting essentially of” refers to those elements required for a given embodiment. The term permits the presence of elements that do not materially affect the basic and novel or functional characteristic(s) of that embodiment of the invention.

The terms “disease”, “disorder”, or “condition” are used interchangeably herein, refer to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, or affectation.

As used herein, the term “cardiovascular disease” or “CVD,” generally refers to heart and blood vessel diseases, including, but not limited to, atherosclerosis, coronary heart disease, cerebrovascular disease, microvascular disease (e.g. renal and nerve damage), and peripheral vascular disease. Cardiovascular disorders are acute manifestations of CVD and include, but are not limited to, myocardial infarction, stroke, angina pectoris, transient ischemic attacks, and congestive heart failure. Cardiovascular disease, including atherosclerosis, usually results from the buildup of fatty material, inflammatory cells, extracellular matrix and plaque. Clinical symptoms and signs indicating the presence of CVD include one or more of the following: chest pain and other forms of angina, shortness of breath, sweatiness, Q waves or inverted T waves on an EKG, a high calcium score by CT scan, at least one stenotic lesion on coronary angiography, or heart attack.

The term “biological sample” as used herein denotes a sample taken or isolated from a biological organism, e.g., an animal or human. Exemplary biological samples include, but are not limited to, a biofluid sample; a body fluid sample, blood (including whole blood); serum; plasma; urine; saliva; a biopsy and/or tissue sample etc. The term also includes a mixture of the above-mentioned samples. The term “biological sample” also includes untreated or pretreated (or pre-processed) biological samples. In some embodiments, a sample can comprise one or more cells from a subject.

The biological sample can be obtained by removing a sample from a subject, but can also be accomplished by using previously isolated samples (e.g. isolated at a prior time point and isolated by the same or another person). In addition, the biological sample can be freshly collected or a previously collected sample.

The terms “lipoprotein” and “lipoprotein particle” as used herein refer to particles obtained from blood (e.g., mammalian blood) which include apolipoproteins biologically assembled with noncovalent bonds to package for example, without limitation, cholesterol and other lipids. Lipoproteins preferably refer to biological particles having a size range of about 7 to 1,000 nm, and include VLDL (very low density lipoproteins), IDL (intermediate density lipoproteins), LDL (low density lipoproteins), Lp(a) [lipoprotein (a)], HDL (high density lipoproteins) and chylomicrons.

“Nanoparticle”, “microparticle” and “particle” means material of biological, organic, or inorganic origin having a covalent or non-covalently bound assembly of molecules ranging in size from nanometer (nanoparticles) to micrometer (microparticle) to even larger size ranges.

As used herein, the term “high density lipoprotein” or “HDL” includes protein or lipoprotein complexes with a density from about 1.06 to about 1.21 g/mL. HDL is known to contain two major proteins, Apolipoprotein A-I (ApoA-I) and Apolipoprotein A-II (ApoA-II); therefore, in some embodiments, the term “HDL” also includes an ApoA-I and/or an ApoA-II containing protein or lipoprotein complex.

As used herein, the terms “HDL particles” or “HDL-P” refer to a population of HDL particles. In some embodiments, “HDL particles” can mean all HDL particles regardless of type or size. In some embodiments, “HDL particles” can mean one or more subpopulations of HDL particles, which will generally be clear from context. The number of subpopulations can vary depending upon the particular classification. For example, HDL particles can be classified into five subpopulations as described herein: very small HDL particles, small HDL particles, medium HDL particles, large HDL particles, and very large HDL particles. It should be noted that this classification is different from that in Rosenson et al., Clinical Chemistry 2011, 57:3, 392-410. Other classification systems can be used.

As used herein, the terms “concentration of HDL particles” and “level of HDL particles” are used interchangeably.

As used herein, the terms “very small HDL particles” or “VS-HDL particles” refer to HDL particles having a size of less than 8 nm.

As used herein, the terms “small HDL particles” or “S-HDL particles” refer to HDL particles having a size in the range of 8 nm to less than 8.5 nm.

As used herein, the terms “medium HDL particles” or “M-HDL particles” refer to HDL particles having a size in the range of 8.5 nm to less than 9.9 nm.

As used herein, the terms “large HDL particles” or “L-HDL particles” refer to HDL particles having a size in the range of 9.9 nm to less than 11.5 nm.

As used herein, the terms “very large HDL particles” or “VL-HDL particles” refer to HDL particles having a size of at least 11.5 nm.

As used herein, the terms “subpopulations” and “subspecies” are used interchangeably.

As used herein, a “subject” means a human or animal. Usually the animal is a vertebrate such as, but not limited to a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female. Additionally, a subject can be an infant or a child.

Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with CVD. A human subject can be of any age, gender, race or ethnic group. In some embodiments, the subject can be a patient or other subject in a clinical setting. In some embodiments, the subject can already be undergoing treatment.

The term “statistically significant” or “significantly” refers to statistical significance and generally means a two standard deviation (2SD) or greater difference.

As used herein, the term “significantly” should be interpreted as if modified by the term “statistically”.

The singular terms “a,” “an,” and “the” include plural referents unless context clearly indicates otherwise. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages may mean ±1% of the value being referred to. For example, about 100 means from 99 to 101.

Although methods and materials similar or equivalent to those disclosed herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

FIGS. 1A-1E are experimental data showing calibration and validation of IMA.

FIG. 1A is a plot of superimposed IMA size distribution spectra (smoothed) of serial dilutions of glucose oxidase. Total protein concentrations, determined by A₂₈₀, are indicated in the figure; major oligomer species are labeled.

FIG. 1B is a plot showing that duplicate serial dilutions of glucose oxidase were analyzed by IMA and by A₂₈₀. Spectral peak areas were plotted against particle concentrations (calculated from total protein concentration and the oligomer distribution).

FIG. 1C is a plot of combined data after IMA of bovine catalase and human transferrin.

FIGS. 1D-1E are plots showing that serial dilutions of recombinant HDL (FIG. 1D) or gold nanoparticles (FIG. 1E) were quantified by Lowry protein or A₅₂₁, respectively, and by calibrated IMA. Particle concentrations obtained by these orthogonal methods are plotted against each other in (FIG. 1D) and (FIG. 1E). Lines were determined by simple linear regression.

FIGS. 2A-2F are experimental data showing quantitation and characterization of HDL subspecies.

FIGS. 2A-2D show analyses of human HDL size distribution spectra. Solid black traces (nudged +0.5 on the vertical axis, for clarity) are IMA spectra of HDL. Voigt probability distribution curves (nudged +0.25) correspond to the 3 HDL subspecies. The sum of the 3 Voigt curves is also shown. Peak parameters were iteratively adjusted to minimize the residuals (dots). Arrowheads indicate minor subspecies occasionally observed and not independently quantified.

FIGS. 2E-2F are bar graphs showing that isolated HDL (p=1.063-1.21 mg/mL), further separated by size-exclusion chromatography, was quantified by calibrated IMA and subsequently evaluated for cholesterol content or sterol efflux capacity on a per particle basis. In FIG. 2E, the efflux capacity of each fraction was determined in two cellular systems: J774 macrophages (upper panel; Khera, A. V. et al., N. Engl. J. Med. 364, 127-135 (2011)) and ABCA1-expressing baby hamster kidney (BHK) cells. Values are normalized to the fraction showing maximum efflux (dashed line) and represent the means±SEMs of duplicate efflux determinations from two HDL samples (isolated from pooled plasma) fractionated and analyzed on different days. In FIG. 2F, the total cholesterol content (free and esterified) of HDL fractions was determined on a per particle basis. Values are means±SEMs of three independent HDL fractionations.

FIGS. 3A-3D are plots showing HDL-P in control and carotid cerebral vascular disease (CCVD) subjects. In FIG. 3A, HDL particle concentrations were measured in subjects with (n=40) and without (n=40) CCVD. The concentrations of each HDL-subspecies, as well as the total particle concentration, are shown as box plots. In FIGS. 3A & 3B, classic lipid risk factors of CVD are shown in separate panels. P values are from Student's t-tests (2-tailed) comparing subjects with (+) and without (−) CCVD. For all boxplots, center lines show the median, boxes represent the quartiles, and whiskers indicate the range. In FIG. 3D, unadjusted odds ratios (ORs) for HDL-P, as well as classic lipid risk factors, were calculated through logistic regression. Open dots indicate the 95% confidence interval (CI) does not cross one. Results are expressed as OR±95% CI per 1 standard deviation.

FIGS. 4A-4I are experimental data showing relationships of HDL subspecies particle concentrations with HDL-C and apoA-I. Total and subspecies HDL particle concentration versus HDL-C (FIGS. 4A-4D) or apoA-I (FIGS. 4F-4I). The HDL subspecies plotted is indicted in left margin. Linear regressions are shown as thick black lines. Pearson r values are indicated in each panel. In FIG. 4D, subjects with above-average HDL-P and below-average HDL-C are shown as dots within the upper-left quadrant. Subjects with below-average HDL-P and above-average HDL-C are shown as dots within the lower-right quadrant. Dashed boxes delineate the quadrants by mean HDL-P and mean HDL-C. HDL particle concentrations, and HDL-C values, of these two groups are compared in FIG. 4E. Bars indicate means±SEMs; *; P<0.05, **; P<0.01, ***; P<0.001 (two-tailed Student's t-test).

FIGS. 5A-5C are experimental data showing HDL particle concentration in early atherosclerosis. In FIG. 5A, HDL particle concentrations were measured in subjects with and without endothelial dysfunction (ED), a marker of early CVD. The concentrations of each HDL-subspecies, as well as the total particle concentration, are shown as box plots. In FIG. 5B, classic lipid risk factors of CVD are shown in separate panels. P values are from Student's t-tests (2-tailed) comparing subjects with ED (ED:(+)) and without (ED:(−)). For all boxplots, the thick center lines show the median, the box represents the interquartile range, and the whiskers indicate the range. In FIG. 5C, odds ratios (OR) for HDL subspecies as well as classic lipid risk factors were also calculated through logistic regression. Results are expressed as odds ratio per 1 standard deviation.

FIG. 6 is a bar graph showing serum testosterone levels in hypogonadal males at baseline and after testosterone replacement with transdermal gel (gel-T) or oral (oral-T) formulations. Bars represent means±standard deviation. P-values comparing formulation groups were calculated from independent Student's t-tests. P-values comparing on-treatment values to baseline levels were calculated from paired Student's t-tests. All t-tests were two-tailed and uncorrected.

FIG. 7 is a set of graphs showing total and subspecies HDL-P, determined by calibrated IMA, and HDL cholesterol levels (HDL-C) in hypogonadal males undergoing testosterone replacement therapy by oral formulation. Box plots represent the median (center line), interquartile range (boxes) and range (whiskers); outliers are plotted individually. P-values were calculated by paired t-tests comparing measures at a given time point to baseline levels.

FIG. 8 is a set of graphs showing total and subspecies HDL-P, determined by calibrated IMA, and HDL cholesterol levels (HDL-C) in hypogonadal males undergoing testosterone replacement therapy by transdermal gel formulation. Box plots represent the median (center line), interquartile range (boxes) and range (whiskers); certain outliers are plotted individually. P-values were calculated by paired t-tests comparing measures at a given time point to baseline levels.

FIG. 9 is a plot showing that the levels of HDL-cholesterol are significantly lower in HD group than in control group.

FIG. 10 is a set of plots showing that three sizes of HDL particle (HDL-P) were observed (all subjects).

FIG. 11 is a plot showing that the concentrations of medium, large, and total but not small HDL particles are significantly lower in HD group than in control group.

FIG. 12 is a plot showing odds ratios for HD status for HDL-C, and HDL-P concentrations.

FIGS. 13A-13B are bar graphs demonstrating the robustness of calibrated IMA. In FIG. 13A, four independent blood samples were collected in either EDTA- or heparin-containing sample collection vials. In FIG. 13B, in separate experiments plasma was exposed to freeze-thaw cycles, either 1, 2, or 3 cycles from −80° C. to room temperature. For each sample, HDL was isolated and analyzed by calibrated IMA in triplicate. Bars represent means±SDs. No statistical differences in total HDL-P or subspecies HDL-P were found in either experiment.

FIGS. 14A-14B are experimental data showing apparent molecular weights of HDL subspecies by calibrated IMA. In FIG. 14A, the observed diameters of reference proteins were plotted against their molecular weights. A best-fit curve (power series), shown in solid black, was used to interpolate the apparent molecular weight of HDL subspecies. Dashed horizontal lines indicate average diameters of HDL subspecies while vertical solid lines descending from the intersection with the best-fit curve indicate the apparent molecular weight. In FIG. 14B, for each subspecies, the average diameters, approximate size-spans, and their corresponding apparent molecular weights are tabulated.

FIGS. 15A-15C show calibrated IMA data acquisition and analysis. (FIGS. 15A-15C) Schematic of the electrospray differential ion mobility analyzer. In FIG. 15A, in the charge-reducing electrospray source, particles in solution are converted to gas-phase ions—mostly singly-charged anions and cations. In FIG. 15B, the IMA separates singly-charged cations according to their electrophoretic mobilities, which depend largely on particle diameter. In FIG. 15C, selected particles exiting the IMA are enlarged by condensing water and enumerated by laser light scattering in the condensation particle counter (CPC). Size distribution spectra were recorded by ramping the IMA voltage and enumerating particles of known electrophoretic mobilities.

FIG. 16 is a plot showing calibrated IMA spectra for three LCAT-deficient spectra and calibrated IMA spectra for three healthy control subjects. Specifically, FIG. 16 shows that, surprisingly, LCAT-deficient subjects appear to have homogenous α-4 HDLs, whereas the healthy controls have more heterogeneous populations of HDL.

FIGS. 17A-17D are experimental data showing the characterization of HDL-P by calibrated IMA.

FIG. 17A is a schematic of generalized workflow.

FIG. 17B-17D show deconvolution of representative IMA size distribution spectra (B-D). Gray curves are Voigt probability distributions fit to the 3 HDL subspecies (labeled). Residuals (differences between the sum of the 3 Voigt curves (dashed line) and the raw spectra) are shown above the spectra (dots). Coefficients of determination (r²) are indicated.

FIGS. 18A-18F are experimental data showing relationships of HDL particle concentration with HDL-C. HDL-P versus HDL-C plots and linear regressions (FIGS. 18A-18D). Pearson r values are indicated. In (FIG. 18D), dashed boxes delineate quadrants by mean values. HDL-P and HDL-C values (means±SEMs) for subjects in the upper-left and lower-right quadrants are compared in (FIG. 18E). A representative IMA spectrum from each group is shown in (FIG. 18F).

FIGS. 19A-19D are experimental data showing HDL-P in 40 control and 40 carotid cerebrovascular disease (CCVD) subjects. HDL-P values (FIG. 19A) and classic lipid risk factors of cardiovascular disease (FIG. 19B, 19C) are shown as boxplots. Unadjusted odds ratios (ORs), calculated through logistic regression, are expressed as OR±95% confidence interval (CI) per 1-SD (FIG. 19D). Open dots indicate the 95% CI does not cross one.

FIG. 20 is a set of representative IMA spectra from validation studies. The two panels show IMA size distribution spectra from different individuals (Subjects B and D). Traces of the same color represent triplicate samples prepared in parallel (intra-assay). The different colors represent triplicate samples from two distinct batches prepared on different days (inter-assay).

FIG. 21 is a set of images showing immunoblot analysis of apoA-I in HDL and non-HDL fractions. HDLs from the plasma of 4 individuals were isolated. Equal proportions of the top (HDL) and bottom (non-HDL) ultracentrifugation layers were separated by SDS-PAGE and immunoblotted with a polyclonal antibody to apoA-I. Bands were quantified using the “rolling ball” method (Gassmann M, et al., ELECTROPHORESIS. 2009; 30:1845-55). Lanes are labeled with the subject, the fraction analyzed, and the percent of immunoreactive material in the two fractions. Recovery of apoA-I in the HDL fraction was 80±3% (mean±SD).

FIGS. 22A-22B are spectra showing analysis of human and mouse plasma lipoproteins by calibrated IMA. IMA size distribution spectra (5 to 30 nm) of lipoproteins isolated by ultracentrifugation from human (FIG. 22A) and mouse (FIG. 22B; C57B6J genetic background) plasma. Human plasma was from a CLEAR study subject. The protocol described in the Example section was used to isolate and analyze both human and mouse lipoproteins. Magnified versions of the spectra, from 15 to 29 nm, are shown above the original traces. IDL, intermediate density lipoprotein; LDL, low density lipoprotein.

### DETAILED DESCRIPTION

As described briefly earlier, existing ion mobility methods fail to accurately measure the concentration of particles in the solution phase because ionization efficiency and other sources of signal loss are not accounted for. Further, this challenge has not been appreciated by those skilled in the art. For example, Caulfield et al. reported, “The IM method [ . . . ] not only measures particle size accurately on the basis of physical principles, but also directly counts the particles present at each size. This approach thereby provides the only direct measurement of lipoprotein particle size and concentration for each lipoprotein size, from small HDL to large VLDL.” (Caulfield et al., Clinical Chemistry, 2008, 54, 1307-1316). Thus it is clear that those skilled in the art fail to recognize the following: (1) the algorithm used to translate particle counts into aerosol concentration does not account for the efficiency of electrospray ionization (ESI). The generation and transmission of bare ions during ESI is non-quantitative and highly variable; therefore ESI effectiveness must be considered in quantitative assays. And (2) it is unclear if the proportion of singly-charged particles produced by the charge reduction step is constant for biological particles, such as HDL, that vary widely in size, shape, isoelectric point, and composition.

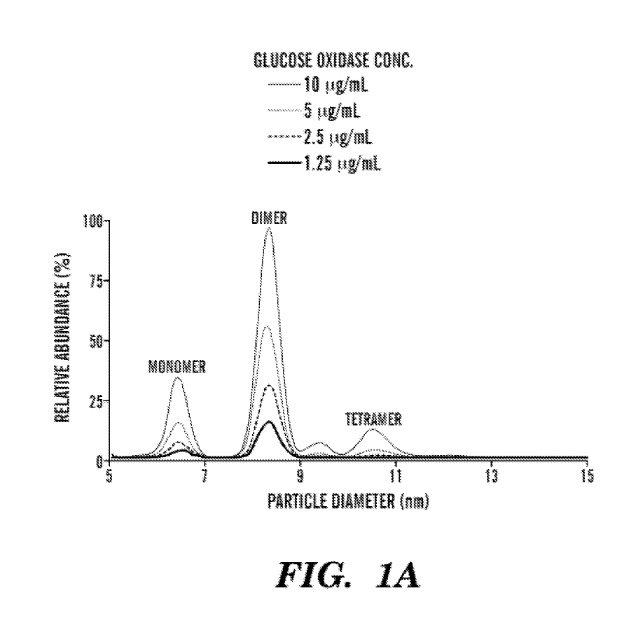

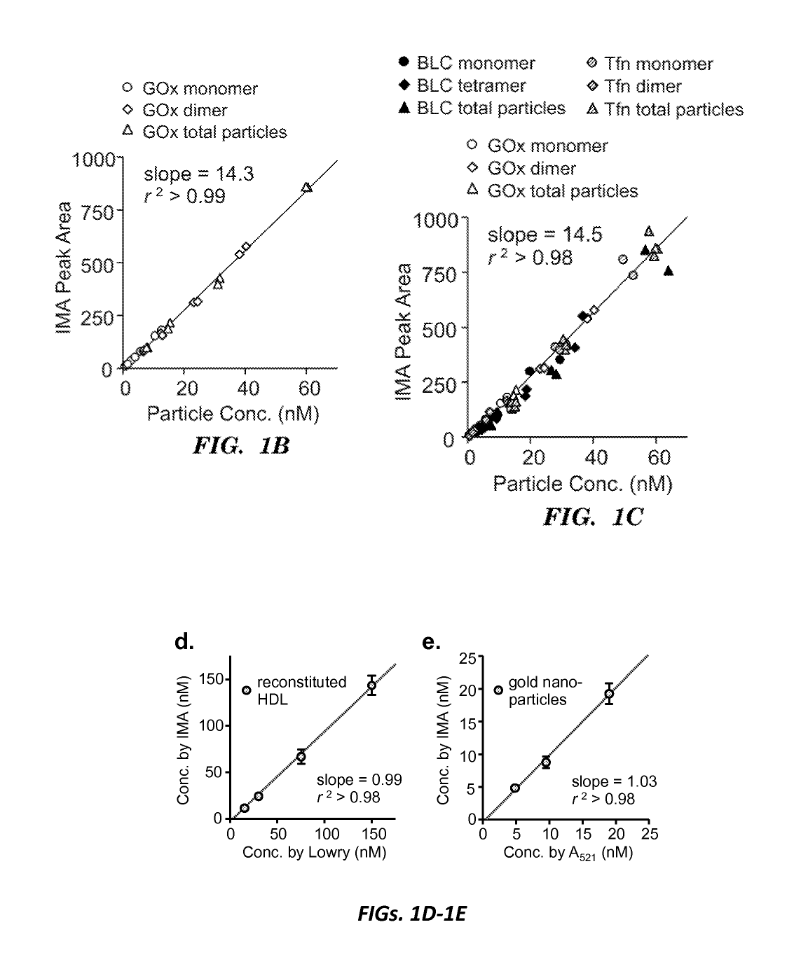

The technology described herein is based, in part, on the surprising discovery that ionization efficiency and other sources of signal loss can be accounted for by a calibration step, where IMA is performed on particles of known solution-phase concentration. It has been surprisingly discovered, among other things, that different particles in solutions—even when they have different diameters or different material properties—elicit similar responses when analyzed by the same instrument (see FIGS. 1B & 1C). Therefore, a calibrant (i.e., a solution comprising reference particles of known concentration) can be used to calibrate an ion mobility analyzer. The calibration curve obtained by this calibration step can be applied to processing the IMA spectrum of a sample solution having unknown particle concentration for the purpose of quantifying the particle concentration.

The methods of calibrated IMA described herein improve upon existing IMA methods. Specifically, a calibration method is provided herein that permits IMA to accurately quantify particle concentrations in solutions, e.g., concentration of HDL-P or subspecies thereof in a biological sample. The methods of calibrated IMA have been validated, and their robustness has been tested. The methods described herein can be used in the characterization of particles in a solution, such as particle concentration and molecular weights of particles. The methods can be particularly useful for the measurement of biological samples, e.g., blood, serum, or urine samples.

### Calibrated IMA

In one aspect, the technology provides a method of characterizing particles in a sample solution, the method comprising: (i) converting a portion of the particles in the sample solution into gas-phase ions; (ii) performing an ion mobility measurement on the gas-phase ions, whereby the gas-phase ions are enumerated according to size, thereby producing data relating particle size to relative abundance; (iii) processing the data by using a calibration regression; and (iv) quantitatively determining particle concentration in the sample solution based on the processing.

The calibration regression can be obtained by first performing steps (i) and (ii) on reference particles of known solution-phase concentration. Stated another way, a portion of the reference particles in a solution are converted into gas-phase ions, and an ion mobility measurement is performed on these gas-phase ions. The calibration regression can then be constructed by relating the total number of enumerated gas-phase ions of the reference particles to the known solution-phase concentration of the reference particles. The calibration regression can be stored, for example, in a computer.

In some embodiments, at least one solution of reference particles (e.g., 1, 2, 3, 4, 5, 6, 7, 8, 9 or more) is used to obtain the calibration regression. When two or more solutions of reference particles are used, the concentrations of these solutions can vary. The concentrations of reference particles should span below and above the range of concentrations observed (or expected) for the particles being characterized (e.g. HDL particles). In some embodiments of reference particles used for HDL particle characterization, the reference particle concentration can be in the range of 1-60 nM. The solutions comprising reference particles can be stored (e.g., frozen at −80° C.) at much higher concentrations and diluted prior to use.

Ion mobility measurements are known in the art and can be performed without deviation from existing methods. Generally, highly charged ions can be largely neutralized by alpha-particles, yielding a small proportion of singly-charged cations, which are introduced into the mobility analyzer. As the ionized particles move through an electromagnetic field, their movement or translation is affected by the electromagnetic field. The ionized particles are subsequently separated according to their electrophoretic mobility and, subsequently, enumerated by a particle counter. Because electrophoretic mobility depends chiefly on size, IMA data can be expressed in terms of particle diameters corresponding to the calculated diameter of a singly-charged, spherical particle with the same electrophoretic mobility.

Particles in a solution can be converted to gas-phase ions through a variety of ionization methods. Suitable forms of ionization include electrospray ionization, nanoelectrospray ionization, matrix-assisted laser desorption ionization (MALDI), laser/light, thermal, electrical, atomized/sprayed and the like, or combinations thereof. It should be noted that it's preferred that the calibrant is ionized using the same method as the sample.

In one embodiment, the ionization method is electrospray ionization. In the charge-reducing electrospray source, particles in solution are converted to gas-phase ions—mostly singly-charged anions and cations. It is important to note that myriad factors influence the generation and transmission of bare ions during ESI including: spray needle position and tip geometry, gas-composition and pressure, liquid and gas flow-rates, analyte composition, solvent properties (such as ionic strength and viscosity), spray needle voltage, orifice voltage (and geometry), conductor compositions, etc.

The particles in a sample solution and reference particles can each be independently selected from the group consisting of biological particles, inorganic particles, metallic particles, metallo-organic particles, organic particles, polymeric particles, and a combination thereof.

As used herein, the term “biological particle” means a material having a covalently or non-covalently bound assembly of molecules derived from a biological source. Examples are apolipoproteins; lipoproteins (e.g., whole HDL, fractionated HDL, whole LDL, fractionated LDL, whole VLDL, fractionated VLDL, or a combination thereof); complexes of apolipoproteins; complexes of lipids with proteins, peptides (e.g., monomeric or oligomeric), nucleic acids or other components; transfer RNA; plasmids; liposomes; lipid droplets; lipoprotein particles assembled from apolipoproteins and lipids or other components (e.g., drugs, siRNA etc.); viral components assembled from lipids, coat proteins and glycoproteins; ribosomes; synthetic peptides and proteins; immune complexes assembled from antibodies and their cognate antigens, etc.; microparticles and other assemblies derived from cells (e.g. ribosomes, mitochondria, exosomes, nuclei, platelets); virus; bacteria; and even entire cells.

Inorganic particles can include, but are not limited to, metallic particles, semiconductor particles, and dielectric particles. Metallic particles can be comprised of any metal such as gold, silver, platinum, copper, iron, aluminum, or an alloy. Semiconductor particles can be comprised of any semiconducting material such as silicon, GaAs, GaP, InAs, InP, CdS, CdSe, and CdTe. Dielectric particles can be comprised of any dielectric material such as silica, metal oxide (e.g., alumina, magnesium oxide, or titanium oxide), and magnesium fluoride.

Without limitations, examples of reference particles include gold nanoparticles, silver nanoparticles, polystyrene nanoparticles, silica nanoparticles, purified proteins such as glucose oxidase, and a combination thereof. Preferably, the solution comprising the reference particles is shelf stable. In some embodiments, the reference particles are of known size.

The size distribution of the reference particles should be appropriately narrow. In some embodiments, the peak width (full-width at hald-max) of the reference particle size distribution should not substantially exceed (by >15%) the resolution of the instrument. The resolution (defined as full-width at half-max of peak/size of peak) of the instrument used for these analyses is approximately 20 at 10 nm.

In some embodiments, the sample solution is an aqueous solution. The aqueous solution can be pretreated prior to ionization, for example, centrifugation, filtration, thawing, purification, dialysis, or combinations thereof. In some embodiments, the aqueous solution can undergo ultracentrifugation. In some embodiments, the aqueous solution can undergo dialysis to substantially remove salts.

In some embodiments, the reference particles are in an aqueous solution.

Generally, IMA can produce a spectrum that relates particle size to relative abundance. In one embodiment, the method further comprises a step of determining the subspecies or subpopulations of the particles in the sample solution. This step is also referred to as deconvolution herein and is used to obtain useful underlying information from a complex spectrum. Specifically, the method further comprises superimposing a plurality of distribution curves over the spectrum, each distribution curve representing a subpopulation of the gas-phase ions according to size, and iteratively adjusting parameters of the distribution curves to minimize the difference between the spectrum and sum of the distribution curves. It should be noted that said superimposing can be done virtually.

A variety of distribution curves can be used. The distribution curve can be a probability distribution curve. Preferably, the distribution curve is continuous and includes a peak. The distribution curve can be symmetrical or asymmetrical. Distribution curves applicable to the present technology include, but are not limited to, a Gaussian, a split Gaussian, a Voigt, a split Voigt, a pseudo-Voigt, a Pearson7, a split Pearson7, a Lorentzian, and a split Lorentzian distribution. In one embodiment, the distribution curve used for curve fitting is a Voigt distribution curve.

Before a plurality of distribution curves are superimposed to the IMA spectrum, the peaks on the spectrum can be determined by the user or software. These peaks can then be used to guide the curve fitting. For example, if n (n=1, 2, 3, 4, 5, 6, 7, 8, 9, or more) peaks are located on the spectrum, n distribution curves can be used for the curve fitting; the position of each peak can be used for the peak position of the corresponding distribution curve. As it is known in the art of data analysis, the spectrum can be smoothened to remove false peaks resulting from noise. In some embodiments, the user can also manually set the number of peaks, for example, based on the knowledge of the particles in the sample solution. For example, if a user is aware that the particles in the sample solution only have three subspecies, three distribution curves are to be used in the curve fitting.

A merit function, also known as a figure-of-merit function, can be used to evaluate the difference between the spectrum and sum of the distribution curves and determine whether the curve fitting is optimal. In one embodiment, the merit function is the sum of squared residuals (SSR), also known as the residual sum of squares or the sum of squared errors of prediction. It is a measure of the discrepancy between the data and an estimation model. A small SSR indicates a tight fit of the model to the data. If the sum of squared residuals is minimized, the curve fitting is considered to be optimal.

Curve fitting using a plurality of distribution curves can be done using existing data-processing software or customized scripts. These data-processing software or scripts include Matlab® by MathWorks, Mathematica® by Wolfram, Igor® by WaveMetrics, Origin® by OriginLab, and Fityk.

In some embodiments, the method can permit the determination of molecular weight of the particles being characterized. In these embodiments, reference particles of known molecular weight are used. When IMA is performed on the reference particles, a regression relating the particle size and molecular weight can be produced. This regression can then be used to determine the molecular weight of the particles being characterized based on their size.

### Diagnostic Methods

The ability to quantify the absolute concentrations of HDL particles in a biological sample permits the determination of whether HDL-P can be a valid clinical metric. Using the calibrated IMA methods described herein, concentrations of HDL particles and/or subpopulations thereof have been correlated with conditions such as LCAT deficiency and cardiovascular diseases. Some aspects and embodiments of the methods described below are thus related to the use of concentrations of HDL particles and/or subpopulations thereof for the diagnosis of conditions such as LCAT deficiency and cardiovascular diseases.

In all aspects of any of the diagnostic methods described herein, the method comprises measuring the size and concentration of HDL particles in a biological sample obtained from the subject. The HDL particles are selected from the group consisting of very small HDL particles, small HDL particles, medium HDL particles, large HDL particles, very large HDL particles, and a combination thereof. For example, the concentration of HDL particles can be the concentration of all types of HDL particles, or the concentration of one or more HDL particle subpopulations.

In all aspects of any of the diagnostic methods described herein, the method further comprises comparing the concentration of HDL particles with a reference level or a reference profile.

In some embodiments of all aspects of any of the diagnostic methods described herein, the reference level can be the average concentration of HDL particles in a population of healthy subjects or a representative subpopulation of healthy subjects. This would be a “normal” level.

In some embodiments of all aspects of any of the diagnostic methods described herein, the reference profile can be the average health profile of HDL particles in a population of healthy subjects or a representative subpopulation of healthy subjects. This would be a “normal” profile. The reference profile can comprise a plurality of values and/or descriptors, each value representing the average level of a subpopulation of HDL particles in a population of healthy subjects or a representative subpopulation of healthy subjects. The reference profile can be present in formats including, but not limited to, a table, a matrix, and a heat map. As a non-limiting example, the reference profile can comprise a first value for VS-HDL particles, a second value for S-HDL particles, a third value for M-HDL particles, a fourth value for L-HDL particles, a fifth value for VL-HDL particles, and a six value for total HDL particles. In another example, the reference profile can comprise a value for VS-HDL particles only.

In some embodiments of all aspects of any of the diagnostic methods described herein, the reference profile can be the average health profile of HDL particles in a representative population of subjects having a particular condition. The particular condition should be the same as the condition that the diagnostic method is intended to diagnose. For example, if the method is intended to diagnose LCAT deficiency, the reference profile can be the average health profile of HDL particles in a representative population of subjects having LCAT deficiency.

A computer system can compare the measured data with the reference profile to determine whether the measured data are consistent or inconsistent with the reference profile. To determine consistency, the measured data can be compared with each value of the reference profile. The measured data are considered to be consistent with the reference profile if they are no more than 10% different, no more than 9% different, no more than 8% different, no more than 7% different, no more than 6% different, or no more than 5% different, from the reference profile.

It should be noted that the reference level or reference profile can be different, depending on factors such as the sample type from which the reference level is derived, gender, age, weight, and ethnicity. Thus, reference levels accounting for these and other variables can provide added accuracy for the methods described herein.

In some embodiments of all aspects of any of the diagnostic methods described herein, the method further comprises determining an odds ratio for the subject based on the measured concentration of HDL particles as compared to a reference level or a reference profile. The odds ratio can be calculated using methods known in the art and the odds ratio can be used to determine the relative risk of the subject developing a particular condition. In some embodiments, the odds ratio can be calculated by using a nominal logistic regression model and adjusted to age using a statistical analysis software.

In some embodiments of all aspects of any of the diagnostic methods described herein, the method further comprises measuring lipoproteins other than HDL. For example, LDL concentrations can be measured to supplement the diagnosis.

In some embodiments of all aspects of any of the diagnostic methods described herein, the size and concentration of HDL particles in the biological sample is measured by the calibrated IMA methods described herein. It should be noted that the data produced by the calibrated IMA methods can include all the information regarding the size and concentrations of all particles and subpopulations thereof. For example, when the concentrations of all HDL particles and VS-HDL particles are of interest, one measurement using the calibrated IMA methods can be sufficient.

In some embodiments of all aspects of any of the diagnostic methods described herein, the biological sample can be blood, plasma, or serum.

Cardiovascular Disease (CVD)

Using the calibrated IMA methods described herein, the cardioprotective effects of HDL particles have been studied. In one aspect, the technology described herein provides a method of determining if a subject is at risk to develop or is suffering from a cardiovascular disease (CVD). In some embodiments, the cardiovascular disease can be selected from the group consisting of atherosclerosis, coronary vascular disease, ischemic heart disease, myocardial infarction, angina pectoris, peripheral vascular disease, cerebrovascular disease, endothelial dysfunction, and stroke.

In some embodiments, the atherosclerosis is selected from the group consisting of coronary artery disease (CAD), carotid cerebrovascular disease (CCVD), and peripheral vascular disease.

In some embodiments of diagnosing atherosclerosis, VS-HDL particles can serve as a clinical metric. Accordingly, in some embodiments of atherosclerosis diagnosis, the method comprises determining that the subject is at risk to develop or is suffering from atherosclerosis if the measured concentration of VS-HDL particles is below the reference level. In some embodiments, the measured concentration of VS-HDL particles is at least 5%, at least 10%, at least 20%, at least 30%, at least 40%, at least 50%, at least 60%, at least 70%, at least 80%, or at least 90% less than the reference level.

In some embodiments of diagnosing atherosclerosis, S-HDL particles can serve as a clinical metric. Accordingly, in some embodiments of diagnosing atherosclerosis, the method comprises determining that the subject is at risk to develop or is suffering from atherosclerosis if the measured concentration of S-HDL particles is below the reference level. In some embodiments, the measured concentration of S-HDL particles is at least 5%, at least 10%, at least 20%, at least 30%, at least 40%, at least 50%, at least 60%, at least 70%, at least 80%, or at least 90% less than the reference level.

In some embodiments of diagnosing atherosclerosis, M-HDL particles can also serve as a clinical metric. Accordingly, in some embodiments of atherosclerosis diagnosis, the method comprises determining that the subject is at risk to develop or is suffering from atherosclerosis if the measured concentration of M-HDL particles is below the reference level. In some embodiments, the measured concentration of M-HDL particles is at least 5%, at least 10%, at least 20%, at least 30%, at least 40%, at least 50%, at least 60%, at least 70%, at least 80%, or at least 90% less than the reference level.

In some embodiments of diagnosing atherosclerosis, total concentration of HDL particles can also serve as a clinical metric. Accordingly, in some embodiments of atherosclerosis diagnosis, the method comprises determining that the subject is at risk to develop or is suffering from atherosclerosis if the measured concentration of all HDL particles is below the reference level. In some embodiments, the measured concentration of all HDL particles is at least 5%, at least 10%, at least 20%, at least 30%, at least 40%, at least 50%, at least 60%, at least 70%, at least 80%, or at least 90% less than the reference level.

In some embodiments of diagnosing atherosclerosis, the method comprises comparing the concentrations of two or more subpopulations of HDL particles with the respective reference levels. In one embodiment, the method comprises comparing the concentrations of VS-HDL particles and S-HDL particles with the respective reference levels. In one embodiment, the method comprises comparing the concentrations of VS-HDL particles and M-HDL particles with the respective reference levels. In one embodiment, the method comprises comparing the concentrations of S-HDL particles and M-HDL particles with the respective reference levels. In one embodiment, the method comprises comparing the concentrations of VS-HDL particles, S-HDL particles, and M-HDL particles with the respective reference levels.

In some embodiments of diagnosing atherosclerosis, the method comprises comparing the concentrations of all HDL particles and at least one subpopulation thereof with the respective reference levels. In one embodiment, the method comprises comparing the concentrations of all HDL particles and VS-HDL particles with the respective reference levels. In one embodiment, the method comprises comparing the concentrations of all HDL particles and S-HDL particles with the respective reference levels. In one embodiment, the method comprises comparing the concentrations of all HDL particles and M-HDL particles with the respective reference levels.

In some embodiments of diagnosing atherosclerosis, the method comprises determining that the subject is at risk to develop or is suffering from atherosclerosis if the measured HDL profile is inconsistent with the reference profile. In these embodiments, the reference profile can be the average health profile of HDL particles in a population of healthy subjects or a representative subpopulation of healthy subjects. By “inconsistent” in this context is meant that, in the profile, one or more subpopulation is significantly greater or less than the respective reference population.

In some embodiments of diagnosing atherosclerosis, the method further comprises prescribing/administering, to the subject determined to have atherosclerosis in this manner, a treatment appropriate for treating atherosclerosis. The current options for the prevention and treatment of atherosclerosis include certain pharmacological approaches, in addition to alteration of lifestyle factors which can ameliorate atherosclerosis, such as diet control, weight loss, increased exercise, and smoking cessation. Examples of pharmacological agents in current use for the treatment and prevention of atherosclerosis are hydroxymnethylglutaryl-coenzyrne A (HMGCoA) reductase inhibitors (statins), nicotinic acid, and fibric acid derivatives. Adjunctive pharmacological treatment includes measures directed toward control of diabetes mellitus and hypertension.

The calibrated IMA methods described herein also provide insights on how HDL particles are correlated with endothelial dysfunction. Specifically, it was discovered that M-HDL particles can serve as a clinical metric for endothelial dysfunction. Accordingly, in some embodiments of diagnosing endothelial dysfunction, the method comprises determining that the subject is at risk to develop or is suffering from endothelial dysfunction if the concentration of M-HDL particles is below a reference level.

Existing testing or diagnosis for endothelial dysfunction can be used to supplement the diagnosis. Current diagnostic methods for endothelial dysfunction include, but are not limited to, angiography with acetylcholine injection, flow mediated dilation as measured by Brachial Artery Ultrasound Imaging (BAUI), and reactive hyperemia index as measured by Itamar Medical's EndoPAT.

In some embodiments of diagnosing endothelial dysfunction, the method further comprises prescribing/administering, to the subject determined to have endothelial dysfunction, a treatment appropriate for treating endothelial dysfunction. Endothelial function can be improved significantly by exercise, smoke cessation, weight loss in overweight or obese persons, and improved diet. Pharmacological interventions to improve endothelial function include, but are not limited to, statins, and renin angiotensin system inhibitors such as ACE inhibitors and angiotensin II receptor antagonists.

LCAT Deficiency

In one aspect, the technology described herein provides a method of determining if a subject has lecithin-cholesterol acyltransferase (LCAT) deficiency. LCAT deficiency is a genetic condition (the LCAT enzyme is completely or partially defective) which is present from birth in those affected. There are at least two forms of LCAT deficiency: familial LCAT deficiency in which there is complete LCAT deficiency, and fish eye disease in which there is a partial deficiency.

Current diagnosis of LCAT deficiency requires genetic testing for LCAT gene mutation and functional activity. In comparison, the method provided herein only requires a simple blood test.

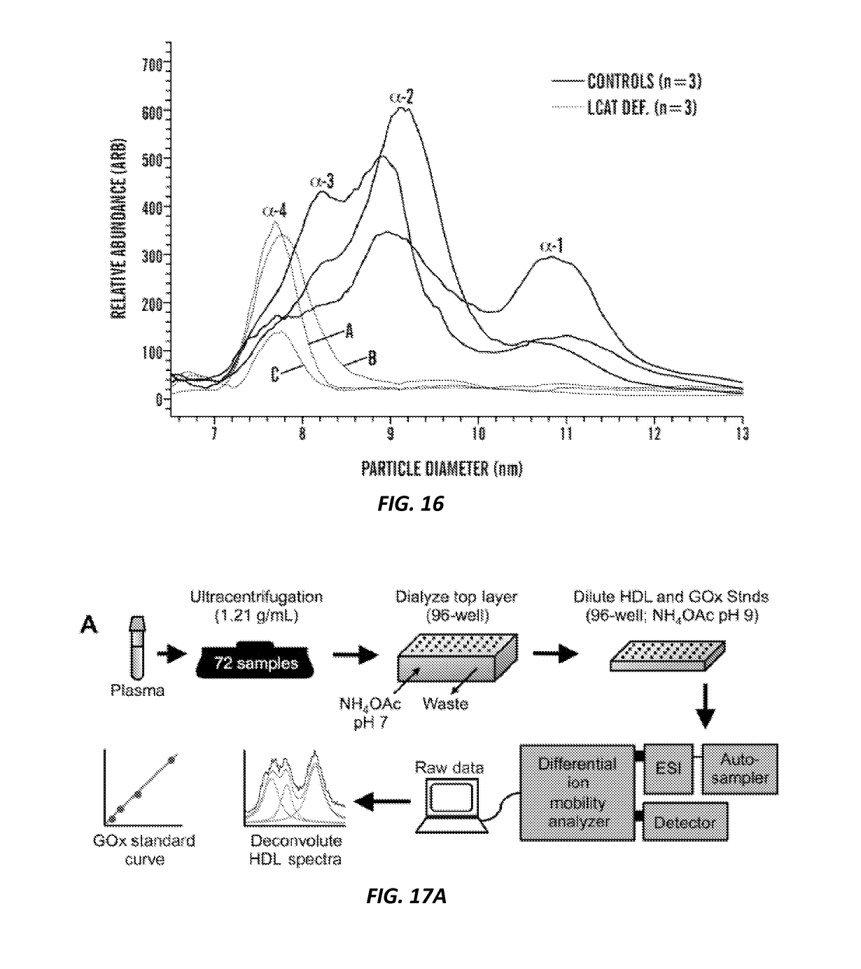

As shown in FIG. 16, it was discovered that within LCAT-deficient subjects, the concentration of VS-HDL particles is at or markedly above the normal level, while other subpopulations of HDL particles are substantially absent. And thus the method comprises determining that the subject has LCAT if the concentration of VS-HDL particles is at or above a first reference level, and the concentration of at least one other subpopulation of HDL particles is below a second reference level.

In some embodiments, the at least one other subpopulation of HDL particles is selected from the group consisting of small HDL particles, medium HDL particles, large HDL particles, very large HDL particles, and a combination thereof.

In some embodiments, the method further comprises administering a treatment appropriate for treating LCAT deficiency. Treatments appropriate for treating LCAT deficiency include, but are not limited to, gene therapies, corneal transplantation, and renal transplantation.

In some embodiments, the first reference level is the average concentration of VS-HDL particles in a population of healthy subjects or a representative subpopulation of healthy subjects. In some embodiments, the second reference level is the average concentration of at least one other subpopulation of HDL particles in a population of healthy subjects or a representative subpopulation of healthy subjects.

Systems

In one aspect, the technology described herein is directed to systems (and computer readable media for causing computer systems) for obtaining data from at least one sample obtained from at least one subject, the system comprising 1) a determination module configured to receive the at least one sample and perform at least one analysis on the at least one sample to determine the level of HDL particles in the sample; 2) a storage device configured to store data output from the determination module; and 3) a display module for displaying a content based in part on the data output from the determination module, wherein the content comprises a signal indicative of the level of HDL particles.

In one embodiment, provided herein is a system comprising: (a) at least one memory containing at least one computer program adapted to control the operation of the computer system to implement a method that includes a determination module configured to measure the level of HDL particles in a test sample obtained from a subject; a storage module configured to store output data from the determination module; a comparison module adapted to compare the data stored on the storage module with a reference level or a reference profile, and to provide a retrieved content, and a display module for displaying the measured level of HDL particles and/or displaying the reference level of HDL particles and (b) at least one processor for executing the computer program.

The term “computer” can refer to any non-human apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; a tablet; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “computer-readable medium” may refer to any storage device used for storing data accessible by a computer, as well as any other means for providing access to data by a computer. Examples of a storage-device-type computer-readable medium include: a magnetic hard disk; a floppy disk; an optical disk, such as a CD-ROM and a DVD; a magnetic tape; a memory chip. The term a “computer system” may refer to a system having a computer, where the computer comprises a computer-readable medium embodying software to operate the computer. The term “software” is used interchangeably herein with “program” and refers to prescribed rules to operate a computer. Examples of software include: software; code segments; instructions; computer programs; and programmed logic.

The computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages.

Embodiments of the systems described herein can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules can perform other functions, thus the modules are not limited to having any particular functions or set of functions.

The functional modules of certain embodiments of the invention include at minimum a measuring module, a storage module, a computing module, and a display module. The functional modules can be executed on one, or multiple, computers, or by using one, or multiple, computer networks. The measuring module has computer executable instructions to provide e.g., levels of expression products etc in computer readable form.

The determination module can comprise any system that can quantitate the absolute concentration of HDL particles in a biological sample. In one embodiment, the determination module is an IMA instrument.

The information determined in the determination module can be read by the storage module. As used herein the “storage module” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon, for example, sample name, biomolecule assayed and the level of said biomolecule. Such information may be provided in digital form that can be transmitted and read electronically, e.g., via the Internet, on diskette, via USB (universal serial bus) or via any other suitable mode of communication.

As used herein, “stored” refers to a process for encoding information on the storage module. Those skilled in the art can readily adopt any of the presently known methods for recording information on known media to generate manufactures comprising expression level information.

In some embodiments of any of the systems described herein, the storage module stores the output data from the determination module. In some embodiments, the storage module stores reference information such as levels of HDL particles in healthy subjects and/or a population of healthy subjects.

The “computing module” can use a variety of available software programs and formats for computing the level of HDL particles. Methods for computing the level of HDL particles are described earlier in this specification. The data analysis tools and equations described herein can be implemented in the computing module of the invention. In one embodiment, the computing module further comprises a comparison module, which compares the level of HDL particles in a sample obtained from a subject as described herein with a reference level or a reference profile. In certain embodiments, the reference level or reference profile can be pre-stored in the storage module. In various embodiments, the comparison module can be configured using existing commercially-available or freely-available software for comparison purpose, and may be optimized for particular data comparisons that are conducted.

The computing and/or comparison module, or any other module of the invention, can include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). In some embodiments users can directly access data (via Hypertext links for example) residing on Internet databases using a HTML interface provided by Web browsers and Web servers.

The computing and/or comparison module provides a computer readable comparison result that can be processed in computer readable form by predefined criteria, or criteria defined by a user, to provide content based in part on the comparison result that may be stored and output as requested by a user using an output module, e.g., a display module.

In some embodiments, the content displayed on the display module can be the level of HDL particles in the sample obtained from a subject. In some embodiments, the content displayed on the display module can be the relative level of HDL particles in the sample obtained from a subject as compared to the average level of HDL particles in a population of healthy subjects. In some embodiments, the content displayed on the display module can indicate whether the subject has an increased likelihood of having or developing atherosclerosis. In some embodiments, the content displayed on the display module can be a numerical value indicating one of these risks or probabilities. In such embodiments, the probability can be expressed in percentages or a fraction. For example, higher percentage or a fraction closer to 1 indicates a higher likelihood of a subject having or developing atherosclerosis. In some embodiments, the content displayed on the display module can be single word or phrases to qualitatively indicate a risk or probability. For example, a word “unlikely” can be used to indicate a lower risk for having or developing atherosclerosis, while “likely” can be used to indicate a high risk for having or developing atherosclerosis.

In one embodiment of the systems described herein, the content based on the computing and/or comparison result is displayed on a computer monitor. In one embodiment of the systems described herein, the content based on the computing and/or comparison result is displayed through printable media. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the computing/comparison result. It should be understood that other modules of the invention can be adapted to have a web browser interface. Through the Web browser, a user can construct requests for retrieving data from the computing/comparison module. Thus, the user will typically point and click to user interface elements such as buttons, pull down menus, scroll bars and the like conventionally employed in graphical user interfaces.

Systems and computer readable media described herein are merely illustrative embodiments of the invention, and therefore are not intended to limit the scope of the invention. Variations of the systems and computer readable media described herein are possible and are intended to fall within the scope of the invention.

The modules of the machine, or those used in the computer readable medium, may assume numerous configurations. For example, function may be provided on a single machine or distributed over multiple machines.

It should be understood that this invention is not limited to the particular methodology, protocols, and reagents, etc., disclosed herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

Although any known methods, devices, and materials may be used in the practice or testing of the invention, the methods, devices, and materials in this regard are disclosed herein.

Some embodiments of the invention are listed in the following numbered paragraphs:

paragraph 1. A method of characterizing particles in a sample solution, the method comprising:
(i) converting a portion of the particles in the sample solution into gas-phase ions;
(ii) performing an ion mobility measurement on the gas-phase ions, whereby the gas-phase ions are enumerated according to size, thereby producing data relating particle size to relative abundance;
(iii) processing the data by using a calibration regression, wherein the calibration regression is obtained by:
(a) performing steps (i) and (ii) on reference particles of known solution-phase concentration; and
(b) constructing the regression relating total number of enumerated gas-phase ions of the reference particles to the known solution-phase concentration;
and
(iv) quantitatively determining particle concentration in the sample solution based on the processing.
paragraph 2. The method of paragraph 1, wherein step (ii) produces a spectrum of particle size distribution.
paragraph 3. The method of paragraph 2, further comprising superimposing a plurality of distribution curves over the spectrum, each distribution curve representing a subpopulation of the gas-phase ions according to size, and iteratively adjusting parameters of the distribution curves to minimize the difference between the spectrum and sum of the distribution curves.
paragraph 4. The method of paragraph 3, wherein the distribution curve is selected from the group consisting of a Gaussian, a split Gaussian, a Voigt, a split Voigt, a Pearson7, a split Pearson7, a Lorentzian, and a split Lorentzian distribution.
paragraph 5. The method of any of the preceding paragraphs, wherein the ion mobility measurement comprises introducing the gas-phase ions into an electromagnetic field having an effect on the translation of the ions, thereby inducing an electrophoretic motion.
paragraph 6. The method of any of the preceding paragraphs, wherein the conversion into gas-phase ions is done by electrospray ionization.
paragraph 7. The method of any of the preceding paragraphs, wherein the particles and reference particles are each independently selected from the group consisting of biological particles, inorganic particles, metallic particles, metallo-organic particles, organic particles, polymeric particles, and a combination thereof.
paragraph 8. The method of paragraph 7, wherein the biological particles are biological cells, proteins or aggregates thereof, or lipoproteins.
paragraph 9. The method of paragraph 8, wherein the lipoproteins are selected from the group consisting of whole HDL, fractionated HDL, whole LDL, fractionated LDL, whole VLDL, fractionated VLDL, and a combination thereof.
paragraph 10. The method of any of the preceding paragraphs, wherein the reference particles comprises nanoparticles selected from the group consisting of gold, silver, polystyrene, silica, purified proteins, and a combination thereof.
paragraph 11. The method of paragraph 10, wherein the purified protein is glucose oxidase.
paragraph 12. The method of any of the preceding paragraphs, wherein the sample solution is an aqueous solution.
paragraph 13. The method of paragraph 12, wherein the aqueous solution is a biological sample.
paragraph 14. The method of paragraph 13, wherein the biological sample is selected from the group consisting of blood, plasma, serum, urine, cerebrospinal fluid, and saliva.
paragraph 15. The method of any of paragraphs 12-14, further comprising dialyzing the aqueous solution to substantially remove salts.
paragraph 16. The method of any of the preceding paragraphs, wherein the reference particles are of known molecular weight.
paragraph 17. The method of paragraph 16, further comprising determining the molecular weight of the particles being characterized.
paragraph 18. The method of any of the preceding paragraphs, wherein the reference particles are of known size.
paragraph 19. A method of determining if a subject is at risk to develop or is suffering from a cardiovascular disease, the method comprising: measuring, in a biological sample obtained from the subject, the size and concentration of HDL particles according to the method of any of paragraphs 1-18.
paragraph 20. The method of paragraph 19, wherein the HDL particles are selected from the group consisting of very small HDL particles, small HDL particles, medium HDL particles, large HDL particles, very large HDL particles, and a combination thereof.
paragraph 21. The method of paragraph 19 or 20, further comprising measuring lipoproteins other than HDL.
paragraph 22. The method of any of paragraphs 19-21, wherein the cardiovascular disease is selected from the group consisting of atherosclerosis, coronary vascular disease, ischemic heart disease, myocardial infarction, angina pectoris, peripheral vascular disease, cerebrovascular disease, endothelial dysfunction, and stroke.
paragraph 23. The method of any of paragraphs 19-22, wherein the biological sample is selected from the group consisting of blood, plasma, and serum.
paragraph 24. The method of any of paragraphs 19-23, wherein the subject is a mammal.
paragraph 25. The method of paragraph 24, wherein the mammal is a human.
paragraph 26. A method of determining if a subject has lecithin-cholesterol acyltransferase deficiency (LCAT), the method comprising:
(i) measuring, in a biological sample obtained from the subject, the concentration of HDL particles; and
(ii) determining that the subject has LCAT if the concentration of very small HDL particles is at or above a first reference level, and the concentration of at least one other subpopulation of HDL particles is below a second reference level.
paragraph 27. The method of paragraph 26, further comprising measuring the size of HDL particles.
paragraph 28. The method of paragraph 26 or 27, wherein the size and concentration of HDL particles are measured according to the method of any of paragraphs 1-18.
paragraph 29. The method of any of paragraphs 26-28, wherein the at least one other subpopulation of HDL particles is selected from the group consisting of small HDL particles, medium HDL particles, large HDL particles, very large HDL particles, and a combination thereof.
paragraph 30. The method of any of paragraphs 26-29, wherein when the concentration of very small HDL particles is at or above the first reference level and the concentration of at least one other subpopulation of HDL particles is below a second reference level, the method further comprises administering a treatment appropriate for treating LCAT.
paragraph 31. The method of any of paragraphs 26-30, further comprising measuring lipoproteins other than HDL.
paragraph 32. The method of any of paragraphs 26-31, wherein the biological sample is selected from the group consisting of blood, plasma, and serum.
paragraph 33. The method of any of paragraphs 26-32, wherein the subject is a mammal.
paragraph 34. The method of paragraph 33, wherein the mammal is a human.
paragraph 35. The method of any of paragraphs 26-34, wherein the first reference level is a concentration of very small HDL particles in a population of healthy subjects.
paragraph 36. The method of any of paragraphs 26-35, wherein the second reference level is a concentration of at least one other subpopulation of HDL particles in a population of healthy subjects.
paragraph 37. A method of determining if a subject is at risk to develop or is suffering from atherosclerosis, the method comprising:
(i) measuring, in a biological sample obtained from the subject, the concentration of HDL particles; and
(ii) determining that the subject is at risk to develop or is suffering from atherosclerosis if the concentration of HDL particles is below a reference level.
paragraph 38. The method of paragraph 37, further comprising measuring the size of HDL particles.
paragraph 39. The method of paragraph 37 or 38, wherein the atherosclerosis is selected from the group consisting of coronary artery disease (CAD), carotid cerebrovascular disease (CCVD), and peripheral vascular disease.
paragraph 40. The method of any of paragraphs 37-39, wherein the size and concentration of HDL particles are measured according to the method of any of paragraphs 1-18.
paragraph 41. The method of any of paragraphs 37-40, wherein the HDL particles are very small HDL particles.
paragraph 42. The method of any of paragraphs 37-40, wherein the HDL particles are medium HDL particles.
paragraph 43. The method of any of paragraphs 37-40, wherein the HDL particles are total HDL particles.
paragraph 44. The method of any of paragraphs 37-43, wherein when the concentration of HDL particles is below the reference level, the method further comprises administering a treatment appropriate for treating atherosclerosis.
paragraph 45. The method any of paragraphs 37-44, wherein the reference level is a concentration of HDL particles in a population of healthy subjects.
paragraph 46. The method of any of paragraphs 37-45, further comprising measuring lipoproteins other than HDL.
paragraph 47. The method of any of paragraphs 37-46, wherein the biological sample is selected from the group consisting of blood, plasma, and serum.
paragraph 48. The method of any of paragraphs 37-47, wherein the subject is a mammal.
paragraph 49. The method of paragraph 48, wherein the mammal is a human.
paragraph 50. A method of determining if a subject is at risk to develop or is suffering from endothelial dysfunction, the method comprising:
(i) measuring, in a biological sample obtained from the subject, the concentration of HDL particles; and
(ii) determining that the subject is at risk to develop or is suffering from endothelial dysfunction if the concentration of HDL particles is below a reference level.
paragraph 51. The method of paragraph 50, further comprising measuring the size of HDL particles.
paragraph 52. The method of paragraph 50 or 51, wherein the HDL particles are medium HDL particles.
paragraph 53. The method of any of paragraphs 50-52, wherein the size and concentration of HDL particles are measured according to the method of any of paragraphs 1-18.
paragraph 54. The method of any of paragraphs 50-53, wherein when the concentration of medium HDL particles is below the reference level, the method further comprises administering a treatment appropriate for treating endothelial dysfunction.
paragraph 55. The method of any of paragraphs 50-54, further comprising measuring lipoproteins other than HDL.
paragraph 56. The method of any of paragraphs 50-55, wherein the biological sample is selected from the group consisting of blood, plasma, and serum.
paragraph 57. The method of any of paragraphs 50-56, wherein the subject is a mammal.
paragraph 58. The method of paragraph 57, wherein the mammal is a human.
paragraph 59. The method of any of paragraphs 50-58, wherein the reference level is a concentration of HDL particles in a population of healthy subjects.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology disclosed herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are disclosed herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments disclosed herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The technology disclosed herein is further illustrated by the following examples which in no way should be construed as being further limiting.

### Example 1

### Quantification of HDL Particle Concentration by Calibrated Ion Mobility Analysis

Calibrated IMA can Quantify Proteins with Different Molecular Weights (MWs) and Isoelectric Points (pIs).

The linearity of the ion mobility response was first tested by analyzing serial dilutions of highly purified glucose oxidase (MW_{dimer}, 160,000; pI, 4.2) (FIG. 1A). IMA spectral peak areas of glucose oxidase oligomers (e.g., monomer and dimer) were plotted against particle concentrations calculated from the total protein concentration determined by A₂₈₀ (FIG. 1B). A linear (r²>0.99), concentration-dependent response was observed for the dimer, the monomer, and total particle concentration.

To investigate the effects of particle size and physiochemical properties (e.g., pI) on instrument response, two additional proteins were interrogated in the same manner. IMA of serial dilutions of bovine catalase (MWₜₑₜᵣₐₘₑᵣ, 240,000; pI, 5.6) and human transferrin (MWₘₒₙₒₘₑᵣ 80,000; pI, 6.2-6.6) both yielded linear, concentration-dependent responses similar to those obtained with glucose oxidase. Importantly, all three proteins produced calibration curves with essentially equivalent slopes and y-intercepts, which passed near the origin. Indeed a single regression line, fit to the superimposed data (FIG. 1C), had an r²=0.98 and a y-intercept near zero.

These observations indicate that proteins of different molecular weights, oligomeric distributions, and isoelectric points all produced similar instrument responses. For routine analyses, glucose oxidase was used as the working calibrant due to its convenient particle diameter near the center of the HDL size-distribution and its stability in aqueous solution.

Calibrated IMA can Quantify the Absolute Concentration of Reconstituted HDL and Gold Nanoparticles.

Reconstituted, discoidal HDL (9.6 nm diameter) was next used to determine whether calibrated IMA could accurately quantify HDL-P. These particles were selected because they are similar to native HDL and because they contain two apoA-I molecules per particle^{22, 23}, allowing one to establish the concentration of stock solutions based on their protein content. When particle concentrations determined by calibrated IMA were plotted against concentrations calculated from total protein, the data were linear (r²=0.98) and had a slope essentially equal to one (FIG. 1D). Gold nanoparticles (˜10 nm diameter) whose concentration was also determined by absorbance at 521 nm were similarly quantified. Once again, two orthogonal methods yielded nearly identical results for particle concentration (FIG. 1E).

Calibrated IMA can Quantify Three Subspecies of HDL in Human Plasma.

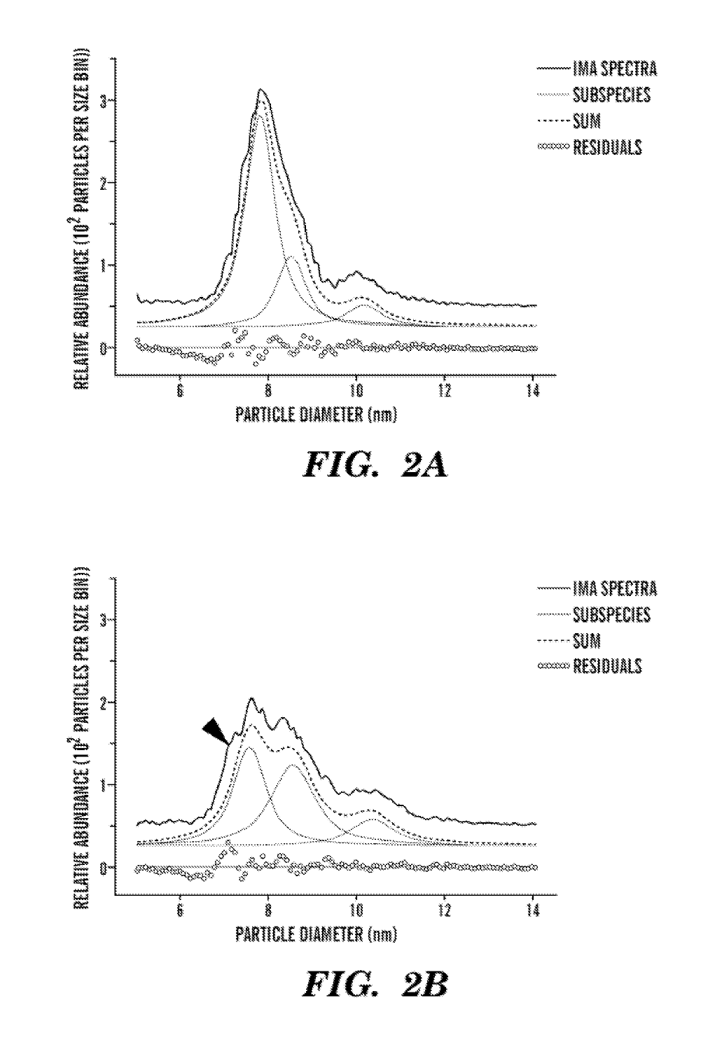

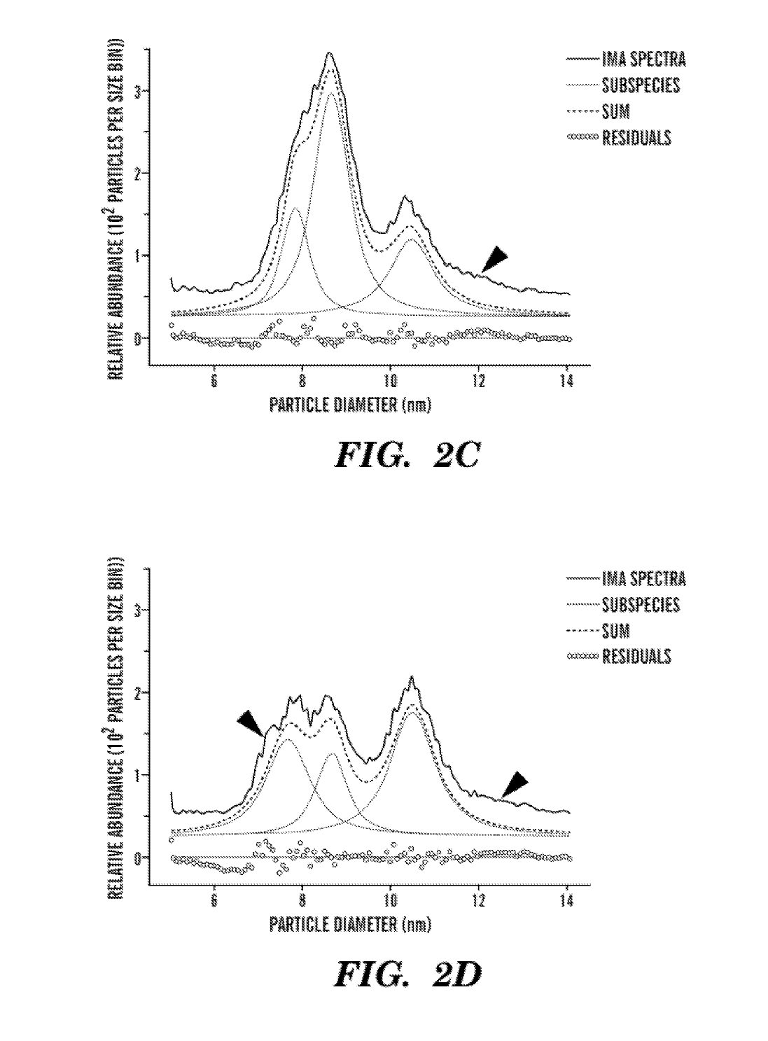

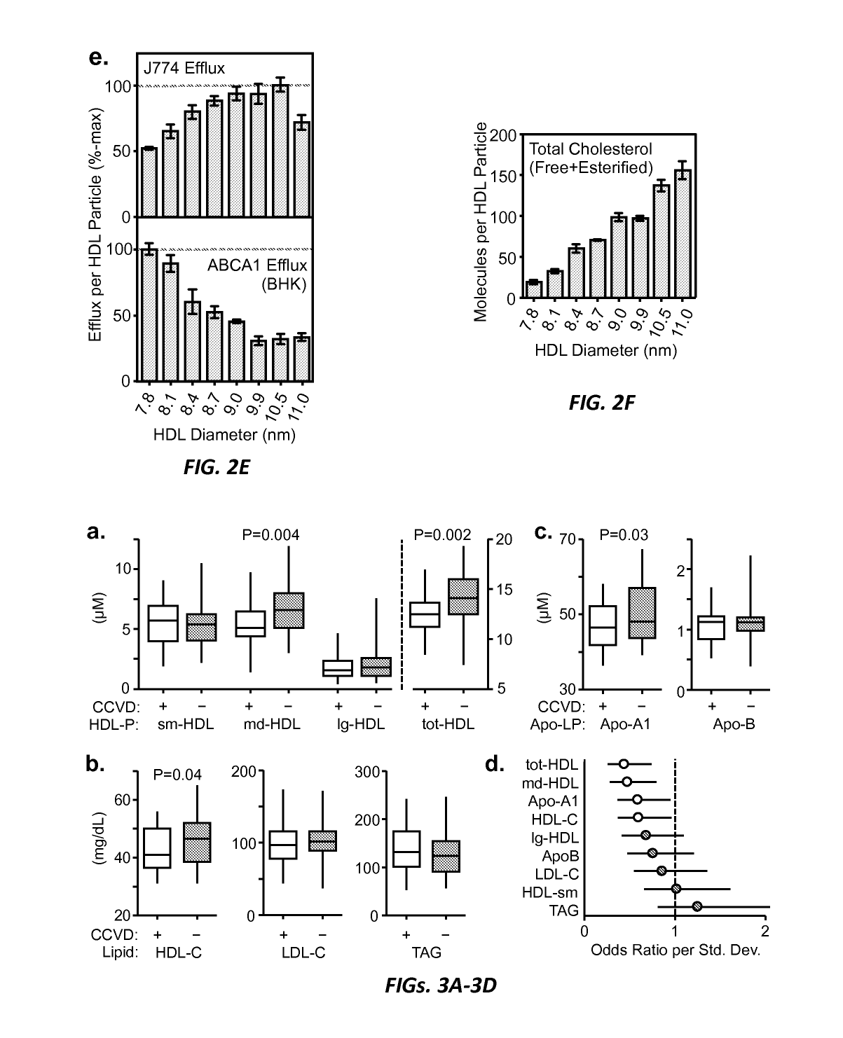

To analyze human HDL, total lipoproteins were isolated from plasma by a single ultracentrifugation spin (p=1.21 g/ml) and dialysis of the preparation to remove salts (which interfere with IMA). The size and concentration of HDL particles were then determined in 80 independent clinical samples (Table 1). Four representative analyses are shown in FIGS. 2A-2D. The raw spectra (solid black traces) indicate particles per size bin. For each spectrum, three HDL subspecies (small, medium, large) were deconvoluted by unsupervised, iterative curve-fitting (curves representing subspecies). It was empirically determined that Voigt curves fit these raw spectra best (typically r²>0.98), though other probability distributions (e.g., Gaussian) also fit well. Finally, peak areas were directly converted to HDL-P by applying the calibration curve. When the same HDL preparation was repeatedly analyzed (nn=6), total HDL-P coefficient of variation (CV) was <6% and the proportion of subspecies was consistent (CV's<10%). When plasma samples (n=12) were subjected to multiple independent isolations and analyses (n=3), intra-assay CV was <7%, and inter-assay CV was <12%.

TABLE 1
HDL-P, Lipids and Demographics of the Clinical Population

| | All Subjects | | Control Subjects | | CCVD Subjects | | | |
|---|---|---|---|---|---|---|---|---|
| | (N = 80) | | (N = 40) | | (N = 40) | | % Change | |
| | mean | SD | mean | SD | mean | SD | (cnt. − cvd) | p-value * |
| HDL-P (uM) | | | | | | | | |
| total | 13.40 | 2.35 | 14.21 | 2.44 | 12.58 | 1.98 | 11.5 | 0.002 |
| small | 5.47 | 1.87 | 5.44 | 1.92 | 5.5 | 1.84 | −1.1 | 0.88 |
| medium | 5.96 | 2.03 | 6.6 | 2.11 | 5.32 | 1.74 | 19.4 | 0.004 |
| large | 1.96 | 1.23 | 2.17 | 1.45 | 1.76 | 0.94 | 18.9 | 0.13 |
| HDL-P composition (%) | | | | | | | | |
| % small | 41.55 | 14.38 | 39.04 | 13.75 | 44.06 | 14.73 | −12.9 | 0.12 |
| % medium | 44.19 | 11.47 | 46.39 | 11.84 | 41.98 | 10.79 | 9.5 | 0.08 |
| % large | 14.26 | 7.51 | 14.57 | 7.94 | 13.95 | 7.15 | 4.3 | 0.71 |
| HDL-size (nm) | | | | | | | | |
| small | 7.85 | 0.15 | 7.85 | 0.14 | 7.85 | 0.16 | 0.0 | 0.85 |
| medium | 8.64 | 0.17 | 8.61 | 0.15 | 8.67 | 0.17 | −0.7 | 0.11 |
| large | 10.35 | 0.65 | 10.35 | 0.17 | 10.35 | 0.16 | 0.0 | 0.99 |
| Lipids | | | | | | | | |
| HDL-C (mg/dL) | 44.7 | 8.7 | 46.7 | 9.6 | 42.67 | 7.39 | 8.6 | 0.04 |
| LDL-C (mg/dL) | 100.1 | 26.2 | 101.85 | 25.46 | 98.35 | 27.2 | 3.4 | 0.55 |
| TAG (mg/dL) | 131.8 | 50.2 | 126.05 | 46.73 | 137.55 | 53.34 | −9.1 | 0.31 |
| apoA-1 (uM) | 48.8 | 7.1 | 50.21 | 7.81 | 46.85 | 5.9 | 6.7 | 0.03 |
| apoB (uM) | 1.12 | 0.30 | 1.145 | 0.339 | 1.071 | 0.26 | 6.5 | 0.28 |
| Demographics | | | | | | | | |
| Age (yr) | 62.58 | 6.18 | 60.59 | 5.19 | 64.6 | 6.5 | −6.6 | 0.003 |
| Sex (% male) | 90 | | 87.5 | | 92.5 | | −5.7 | 0.71 * |

* Student's t-test except “Sex” is Fisher's test

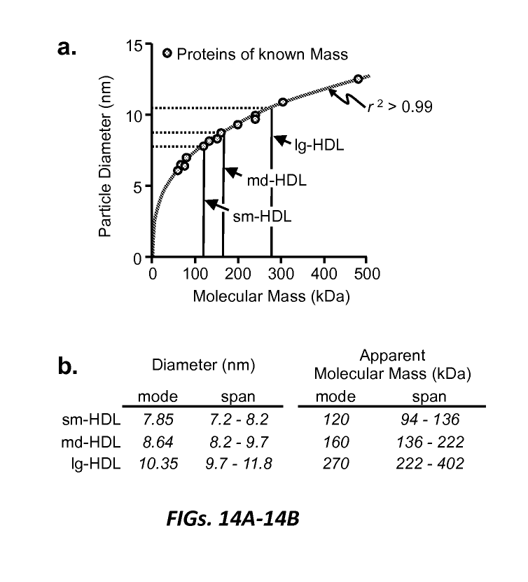

These observations show that calibrated IMA can resolve and reproducibly quantify 3 major HDL subspecies, termed small HDL (sm-HDL), medium HDL (md-HDL), and large HDL (lg-HDL) in human plasma. Their average diameters were: 7.85 nm (small), 8.64 nm (medium), and 10.35 nm (large). By first calibrating the instrument with proteins of known MW, the apparent molecular weights (FIGS. 14A-14B) of the HDL subspecies were also determined by IMA; sm-, md- and lg-, HDL were 120,000, 160,000 and 270,000, respectively. Additional HDL subspecies were observed in certain samples (arrowheads FIGS. 2B, 2C, 2D).

The data revealed remarkable biological diversity in HDL-P, highlighted by striking differences in the proportions of HDL subspecies. While certain samples were composed almost entirely of sm-HDL (FIG. 2A), others showed a majority of lg-HDL (FIG. 2D). Most spectra showed subspecies distributions between these extremes (FIGS. 2B, 2C). The average composition was 42% small-, 44% medium-, and 14% large-HDL. While the relative abundance of HDL subspecies varied dramatically, particle diameters were remarkably consistent: the subspecies CVs were each <3%. HDL-P results are tabulated in Table 2.

TABLE 2
Precision of Calibrated IMA

| | Analytcial^{a} | Inter-assay^{b} | Intra-assay^{b} |
|---|---|---|---|
| | CV (%) | CV (%) | CV (%) |
| Determination | | | |
| Samples | 1 | 12 | 12 |
| Analyses/sample | 6 | 3 | 3 |
| HDL-P | | | |
| total | 5.8 | 6.2 | 11.4 |
| small | 11.9 | 18.8 | 19.7 |
| medium | 5.9 | 12.8 | 15.0 |
| large | 8.0 | 7.1 | 19.8 |
| HDL-P composition | | | |
| % small | 6.6 | 20.1 | 20.6 |
| % medium | 3.0 | 9.4 | 10.2 |
| % large | 9.9 | 6.2 | 10.1 |
| HDL-size | | | |
| small | 0.6 | 1.0 | 1.3 |
| medium | 0.4 | 0.7 | 1.2 |
| large | 0.7 | 0.7 | 1.1 |

^{a}repeated analysis of a single HDL isolate
^{b}repeated isolations and anayses of indepedant plasma samples

Different HDL Subspecies Vary in their Ability to Promote Sterol Efflux by Different Pathways.

Recent studies suggest that HDL's ability to accept cholesterol from J774 macrophages better identifies CVD subjects than does HDL-C level²⁴. To assess the sterol efflux efficiency of HDL subspecies on a per particle basis, HDL was isolated from pooled plasma samples by ultracentrifugation and further fractionated by high-resolution size-exclusion chromatography. Individual fractions were analyzed by calibrated IMA to determine mean particle diameter and particle concentration. Sterol efflux capacity and cholesterol content were then determined on a per particle basis. Efflux from two cell lines, cAMP-stimulated J744 macrophages and transgenic BHK cells induced to express ABCA1, was measured to evaluate different mechanisms of cholesterol transfer.

Sterol efflux from the J774 cells was more efficient with larger (9-10 nm diameter) HDL particles (FIG. 2E), which already contained large amounts of cholesterol (FIG. 2F). In contrast, ABCA1-specific efflux from BHK cells was 3-fold more efficient with small (7.8 nm diameter), cholesterol-depleted HDL particles (FIG. 2E).

HDL-P Independently Associates with Carotid Cerebral Vascular Disease.

To explore whether calibrated IMA might be a clinically useful alternative to HDL-C measurements, HDL-P in control subjects (n=40) was compared with subjects with carotid cerebral vascular disease (CCVD; n=40), a major risk factor for stroke. The latter either had >80% unilateral or bilateral stenosis of the carotid arteries (as documented by ultrasound or MRI) or had undergone a carotid endarterectomy (see ref. 25). The control subjects were free of CVD symptoms, had no prior history of atherosclerotic disease, and had <15% carotid stenosis bilaterally as assessed by ultrasound. The subjects' characteristics are summarized in Table 1.

Compared to the controls, subjects with carotid disease had significantly lower levels of HDL-C, apoA-I, and total HDL-P (P=0.04, 0.03 and 0.002, respectively) (FIG. 3). Unadjusted odds ratios (FIG. 3D) revealed total HDL-P and md-HDL-P were the strongest predictors of CCVD, followed by HDL-C and apoA-I; no other lipid risk factors were significant predictors in this population. Importantly, differences in total HDL-P and md-HDL-P remained significant after adjustment for HDL-C (P=0.02 and 0.04, respectively). After adjustment for LDL and triglycerides, HDL-C was no longer significantly different between groups (P=0.06) while both medium and total HDL-P remained strong predictors of CCVD (P=0.003, 0.009, respectively). Adding age and sex to this model did not affect HDL-P significance.

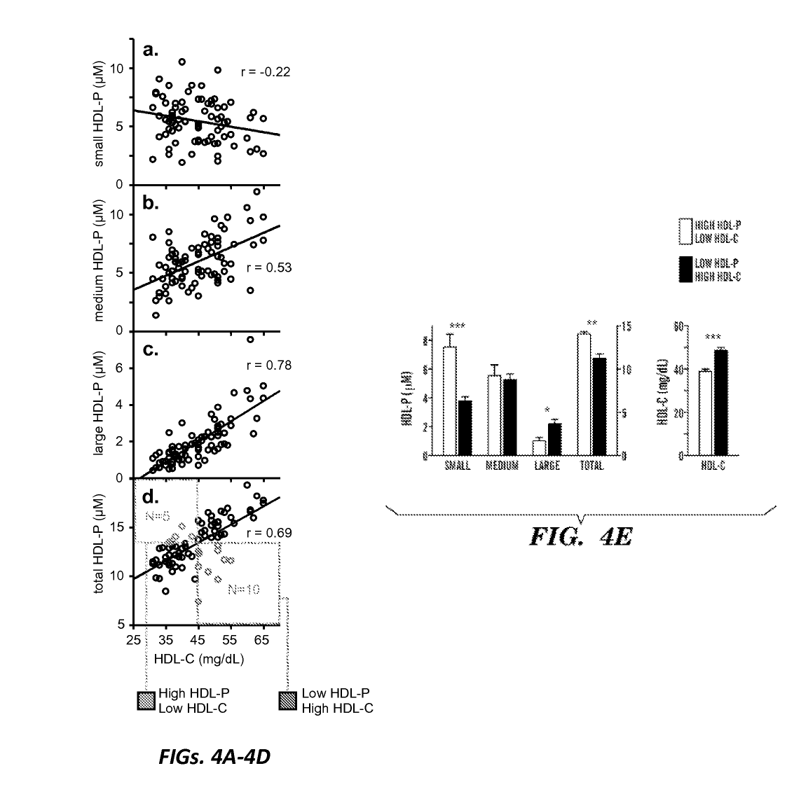

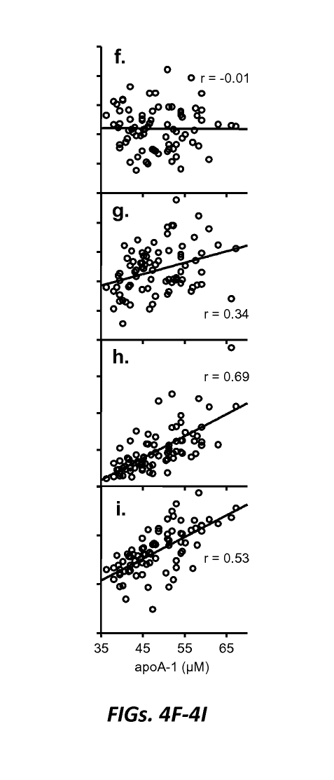

The relationship between HDL-P (total and each subspecies) and HDL-C or apoA-I were next determined in all 80 subjects (FIG. 4). HDL-C predicted >60% of the variance in lg-HDL-P (r=0.78, P<0.0001); lg-HDL-P correlation with apoA-I was also strong (r=0.69, P<0.0001). In contrast, HDL-C predicted <30% of the variance in md-HDL-P (r=0.53, P<0.0001); and md-HDL-P correlation with apoA-I was similarly attenuated (r=0.34, P=0.002). Small HDL concentration did not correlate with HDL-C or apoA-I; moreover, the relationship with HDL-C trended inversely (r=−0.22). Total HDL-P correlations with HDL-C or apoA-I were moderate (r=0.69, P<0.0001 and r=0.53, P=<0.0001, respectively). There was little correlation of HDL-P with level of LDL cholesterol or other lipids (Table 3).

TABLE 3
Correlation matrix: HDL-P and lipids

| | total | small | medium | large | | | | | | | |
|---|---|---|---|---|---|---|---|---|---|---|---|
| | HDL-P | HDL-P | HDL-P | HDL-P | HDL-C | LDL-C | TAG | apoA-1 | apoB | Age | |
| total HDL-P | | 0.099 | <0.001 | <0.001 | <0.001 | 0.818 | 0.927 | <0.001 | 0.859 | 0.243 | P-values |
| small HDL-P | 0.186 | | <0.001 | 0.003 | 0.053 | 0.604 | 0.064 | 0.869 | 0.082 | 0.052 | |
| medium HDL-P | 0.645 | −0.506 | | 0.001 | <0.001 | 0.573 | 0.410 | 0.002 | 0.802 | 0.745 | |
| large HDL-P | 0.567 | −0.330 | 0.355 | | <0.001 | 0.201 | 0.208 | <0.001 | 0.057 | 0.218 | |
| HDL-C | 0.692 | −0.217 | 0.526 | 0.785 | | 0.992 | 0.016 | <0.001 | 0.382 | 0.500 | |
| LDL-C | 0.026 | 0.059 | 0.064 | −0.144 | 0.001 | | 0.746 | 0.355 | <0.001 | 0.315 | |
| TAG | 0.010 | 0.208 | −0.093 | −0.142 | −0.269 | −0.037 | | 0.999 | 0.634 | 0.282 | |
| apoA-1 | 0.669 | 0.019 | 0.341 | 0.688 | 0.814 | −0.105 | <0.001 | | 0.329 | 0.340 | |
| apoB | 0.020 | 0.197 | −0.029 | −0.215 | −0.100 | 0.887 | 0.054 | −0.111 | | 0.345 | |
| Age | −0.132 | −0.218 | −0.037 | 0.139 | 0.076 | −0.114 | −0.122 | 0.108 | −0.108 | | |

Pearson r values

Collectively, these observations indicate that HDL-P can provide clinical information about CVD risk that is independent of other traditional lipid risk factors.

Subspecies Distributions Explain Discordant Values for HDL-P and HDL-C.

HDL-C explained only −50% of the variation in total HDL-P (FIG. 4D). Consistent with this observation, certain subjects showed discordant values of HDL-P and HDL-C, suggesting that subspecies distributions might explain the two metrics' conflicting values. The subset of subjects with high HDL-P (>mean) and low HDL-C (<mean, n=5) was therefore compared with those who had low HDL-P (<mean) and high HDL-C (>mean, n=10) (FIGS. 4D, 4E). The latter had twice the concentration of lg-HDL particles (2.2 vs. 1.0 μM; P=0.02). Conversely, the subjects with high HDL-P/low HDL-C had nearly double the concentration of sm-HDL particles (7.5 vs. 3.8 μM; P=0.0003). Although the two groups had markedly different HDL-C (P=0.0002), they had similar concentrations of md-HDL particles. These observations demonstrate that HDL-P can vary independently of HDL-C because the relative concentration of cholesterol-rich and cholesterol-depleted particles varies significantly among subjects.

Apparent Molecular Weights of HDL Subspecies by IMA

The relationship between particle diameter determined by IMA and by molecular weight (MW) has been extensively studied^{S1-S5}. The correlation is robust, though it can vary slightly between instruments. Therefore, the observed diameters of reference proteins were plotted against their molecular weights (FIG. 14A). A power-series function best fit the data (r²=0.99), as in previous reports of similar analyses^{S5}. Using this curve, the apparent MW of reconstituted HDL was 174,000, in close agreement with MWs determined by other methods^{S6-S8}, suggesting that IMA is a relatively accurate method for determining MW. The apparent MWs of the HDL subspecies were therefore calculated based on average diameters and size range. The apparent MWs of sm-HDL, md-HDL, and lg-HDL were 120,000, 161,000 and 272,000, respectively. Overall, HDL MW ranged from 94,000 to >400,000. These results are comparable to direct measurements of HDL's molecular weight by sedimentation ultracentrifugation^{S9}. Certain samples clearly showed a minor HDL subspecies centered at approximately 12.5 nm and ranging to >13 nm. These particles had diameters corresponding to an apparent MW near 500,000, indicating that HDL particle mass varies up to 5-fold when minor species are considered.

### Principles of Differential Ion Mobility Analysis

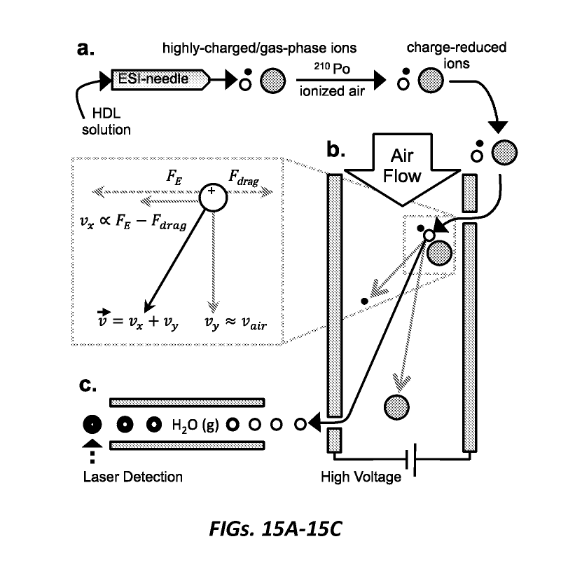

The principles of differential ion mobility, and their application to the analysis of biomolecules, have been extensively reviewed elsewhere^{51,2,10} Briefly, aqueous HDL particles (or other analytes in solution or on a surface) are first converted to highly charged, gas-phase ions by electrospray ionization or other form of ionization (e.g. MALDI). Ions of organic or inorganic form (nanoparticles, microparticles, particles) pass near a²¹⁰Po a-source, where most are neutralized by ionized air (FIG. 15A). The remaining charged species assume a Fuchs charge distribution, which allows the proportion of singly charged cations to be calculated^{S11}. Polydisperse ions then enter the differential ion mobility analyzer, where they quickly assume the velocity of the air moving in the y-direction (FIG. 15B). In the differential mobility analyzer, only singly charged cations are separated according to their electrophoretic mobilities. A particular ion's velocity perpendicular to the laminar airflow is dependent on the force exerted by an electromagnetic field (F_{E}) and the counteracting drag force (F_{drag}). Importantly, drag force is a function of both particle size and shape. Depending on the voltage applied, only particles of a certain electrophoretic mobility successfully traverse the differential mobility analyzer, exit the slit, and enter the condensation particle counter (CPC), where they are detected and quantified. In the CPC (FIG. 15C), particles pass through a chamber of saturated water vapor at 75° C. Condensed water increases the effective diameter of each particle, making it detectable by laser light scattering. Differential mobility analyzer size distribution spectra are generated by scanning the applied voltage while recording the abundance of particles of known electrophoretic mobility. Here, electrophoretic mobilities are expressed as “particle diameter”—corresponding to the calculated diameter of a singly-charged, spherical particle with the same electrophoretic mobility.

Materials: Human serum albumin (A3782), human transferrin (T8158), bovine catalase (C40), Aspergillus niger glucose oxidase (G2133), cholesterol and sodium deoxycholate were obtained from Sigma-Aldrich. Ultrapure human apoA-I was purchased from Academy Biomedical Co. Palmitoyl-oleoyl-phosphatidylcholine was obtained from Avanti Polar Lipids (Alabaster, Ala.). Ammonium acetate, A.C.S. grade (NH₄OAc), and ammonium hydroxide, A.C.S. plus grade (NH₄OH), were obtained from Fisher Scientific. Polyvinylpyrrolidone coated gold nanoparticles (10 nm; NanoXact) were purchased from nanoComposix.

Clinical Population.

All subjects provided signed informed consent, and all protocols were approved by the University of Washington Institutional Review Board. Blood samples were randomly selected from 375 subjects with severe carotid cerebral vascular disease (CCVD) and >1000 controls enrolled in the CLEAR study²⁵. Selection criteria were: age 55 to 80 years, HDL-C 30 to 80 mg/dL, triglycerides <300 mg/dL. CCVD and control subjects were matched by sex and diabetic status. All CCVD subjects had carotid MRI or angiography at a Seattle-area hospital. Subjects with >80% carotid stenosis unilaterally or bilaterally or who had undergone a carotid endarterectomy were considered cases. Control subjects were recruited using clinical databases that excluded anyone with atherosclerosis-related diagnoses. These subjects then underwent a carotid ultrasound. Subjects with <15% carotid stenosis bilaterally were kept as controls. Any symptoms, signs, history, or medical records suggestive of atherosclerotic vascular disease (cardiac or peripheral) were exclusion criteria for control subjects.

HDL Isolation for Calibrated IMA.

Total lipoproteins were isolated from plasma in a single ultracentrifugation step as follows: 50 pL plasma, 50 pL normal saline (with 0.5 mM EDTA), and 130 pL of KBr (p=1.37 g/mL) were added to a thick-wall 7×20 mm ultracentrifugation tube (final p=1.21 mg/mL). Tubes were centrifuged in a 72-position rotor (type 42.2 TI) at 42,000 rpm for 12 h, and 57 pL was taken from the top of each tube and placed in a 96-well constant-flow dialyzer (Spectrum Laboratories Inc.). Samples were dialyzed for 4 h at 4° C. against NH₄OAc (5 mM, adjusted to pH 7.4 with NH₄OH) at a flow-rate of ˜5 mL/min. After dialysis, samples were stored at 0° C. for <24 h before IMA. Immediately prior to analysis, samples were diluted 500-fold (relative to the original plasma volume) with NH₄OAc (5 mM, adjusted to pH 9.2 with NH₄OH). HDL-P analyses by calibrated IMA were not affected by LDL, VLDL, or other lipoproteins.

HDL Isolation and Fractionation for Efflux Studies.

HDL (p=1.063-1.21 g/mL) was isolated from plasma by 2-stage ultracentrifugation³¹. Approximately 500 iug HDL protein was separated by high-resolution size-exclusion chromatography, using fast protein liquid chromatography (FPLC; Supradex 200 column, 0.5 mL flow/min). Typically, 8 HDL size-fractions (0.5 mL) were collected with sufficient material for further analysis. Separations were performed with 150 mM NH₄AOc to limit nonvolatile salt concentration in the samples. The elution profiles of HDL subspecies were essentially the same as those observed with 150 mM Tris-buffered saline.

Sterol Efflux.

After HDL was separated by FPLC, HDL-P was determined for individual fractions by calibrated IMA. Samples were then concentrated 10-fold, using 500 iut 3,000 Da MW cut-off spin-filtration devices. Efflux experiments were based on equal particle concentrations; the protein concentration of each fraction was also measured to ensure that the most dilute samples contained at least 2.5 μg of HDL protein.

J774 Macrophages: Sterol efflux to isolated, fractionated HDL was quantified, using J774 cells exactly as described by Rader and colleagues²⁴. Briefly, J774 cells were radiolabeled with [³H]cholesterol for 24 hours, then stimulated with cyclic-AMP for 24 hours in DMEM. Efflux of [³H]cholesterol was measured after a 2-h incubation with HDL-containing medium. Absolute percent-efflux values were normalized to the FPLC fraction displaying the maximum efflux (%-maximum) to account for variations in the biological activity of different HDL preparations.

ABCA1-expressing baby hamster kidney (BHK) cells: ABCA1-specific sterol efflux to isolated and fractionated HDL was quantified using BHK cells expressing mifepristone-inducible human ABCA1 as described previously³². Briefly, BHK cells were radiolabeled with [³H]cholesterol for 24 h in DMEM. Expression of ABCA1 was induced (or not) by incubating the cells for 20 h with DMEM containing 1 mg/mL fatty acid-free bovine serum albumin and 10 nM mifepristone or vehicle. Efflux of [³H]cholesterol was measured after a 2-hour incubation with HDL-containing medium. ABCA1-dependent cholesterol efflux was calculated as the percentage of total [³H]cholesterol (medium plus cell) released into the medium by mifepristone-treated BHK after subtraction of the value obtained with BHK cells not expressing ABCA1 (no mifepristone treatment). Absolute percent-efflux values were normalized to the FPLC fraction displaying the maximum ABCA1 efflux (%-maximum).

Cholesterol Content Per Particle.

After isolated HDL was fractionated by FPLC, HDL-P in individual fractions was determined by calibrated IMA. Total cholesterol was determined using an Amplex® Red Cholesterol Assay kit (#A12216, Invitrogen Life Technologies).

### Calibrated Ion Mobility Analysis (Calibrated IMA)

Particle Generation, Separation, and Detection: Physical principles of ESI-based differential ion mobility analysis are detailed elsewhere in this Example, also see²⁰.

Instrumentation and Operation: Analyses were performed on a scanning mobility particle sizer spectrometer (TSI Inc., Shoreview, Minn., model 3080N) fitted with a nano-differential mobility analyzer (TSI Inc., model 3085) and a charge-reducing electrospray ionization source (CR-ESI; TSI Inc., model 3480). The differential mobility analyzer scanned particles 5 to 30 nm in diameter in 240 s; default instrument parameters were used. Typical electrospray settings were: voltage 2 kV, CO₂ flow 0.15 L/min, and air-flow 1.5 L/min. Monodisperse particles exiting the differential mobility analyzer were detected by a condensation particle counter (TSI Inc., model 3788). Samples were introduced into the electrospray chamber every 15 min by automated loop injections. To limit cross-contamination, the system was allowed to equilibrate for 10 min after each injection before data acquisition.

Deconvolution of HDL spectra: IMA spectra were expressed in units of aerosol particle concentration per size bin ([number/cm³]/size bin) by means of an algorithm supplied by the instrument manufacturer (Aerosol Instrument Manager®, v9.0.0.0, TSI Inc.)³³. Size distributions of human HDL were further analyzed, using open-source, curve-fitting software (fityk version 1.2.0 for Mac³⁴). Examples of deconvoluted IMA spectral data are shown in FIGS. 2A-2D. Using a custom script, spectra were first smoothed by 10-fold data reduction (FIGS. 2A-2D; solid black traces) and then fitted automatically with 3 Voigt curves corresponding to 3 HDL subspecies termed small HDL (sm-HDL), medium HDL (md-HDL), and large HDL (lg-HDL) (FIGS. 2A-2D). The software iteratively adjusts the peak parameters to minimize the weighted sum of squared residuals, or x² (FIGS. 2A-2D; dots). All peak parameters were unfixed but limited in range. For example, the sm-HDL peak center was limited to 7.8±0.4 nm. The exact script is freely available. Finally, HDL subspecies peak areas were converted into aqueous particle concentrations, using glucose oxidase calibration curves.

Standard Curves of Isolated Proteins: Response curves constructed from different proteins were used to establish the linearity of the differential mobility analyzer response. Standard curves of glucose oxidase (GOx) were generated with each batch of HDL, rHDL, or gold nanoparticles to convert differential mobility analyzer response into aqueous particle concentration.

Solutions of purified protein were prepared gravimetrically in H₂O (approximately 0.5 mg/mL). Exact concentrations were determined by absorbance at 280 nm. Solutions were further diluted in NH₄AOc (5 mM, pH 9.2) prior to IMA. Typically, serial dilutions of glucose oxidase (10-1.25 μg/mL) were used for calibration. Particle concentrations of individual protein oligomers were calculated using the formula:

$${O}_{x} = {P}_{tot} [ \frac{{A}_{x}}{{\sum }_{n = 1}^{i} n \cdot {A}_{n}} ] \qquad Eq . 1$$

where Oₓ is the molar concentration of the oligomer x, Pₜₒₜ is the molar concentration of the monomer calculated from A₂₈₀, Aₓ is the peak area of oligomer x, Aₙ is the peak area of the n^{th} oligomer, n is the order of the n^{th} oligomer, and i is the highest order oligomer observed. This formula accounts for the fact that total particle concentration is different than that determined by A₂₈₀ due to the presence of multiple oligomers.

Clinical Analyses: HDL was isolated from plasma and dialyzed to remove salts as described above. Samples were then diluted and analyzed by IMA. A standard curve of glucose oxidase was generated for each batch of 72 samples. The resulting standard curve was used to convert deconvoluted HDL spectral peak areas into aqueous particle concentrations.

### Calibrated IMA Validation: Accuracy

Reconstituted HDL: Discoidal reconstituted HDL (rHDL) was prepared from human apoA-I, palmitoyl-oleoyl-phosphatidylcholine, and free cholesterol by cholate dialysis, as previously described²³. Particles were then separated by high-resolution size exclusion chromatography (Supradex 200, 0.5 mL flow/min). The protein concentration of the purified rHDL particles (9.6 nm hydrated diameter) was determined by modified Lowry assay (Thermo prod#23240) with the addition of 20 μL of Brij-35 detergent solution (30% w/v in H₂O) to eliminate turbidity. Serial dilutions were prepared (5 mM NH₄OAc, pH 9.2) and quantified by calibrated IMA. For validation of calibrated IMA, duplicate analyses of two independent rHDL preparations were performed (N=4). Particle concentrations were also compared, determined by Lowry assay and calibrated IMA of rHDL prepared at another laboratory and shipped for analyses; again the two measures were similar.

Gold Nanoparticles: Stock solutions of polyvinylpyrrolidone coated-gold nanoparticles (10 nm diameter) were concentrated by centrifugation. Particle concentration of the final solution was determined by absorbance at 521 nm. Serial dilutions were then prepared (5 mM NH4OAc, pH 9.2) and quantified by calibrated IMA. To validate calibrated IMA, duplicate analyses of two independent gold nanoparticle preparations (N=4) were performed.

### Calibrated IMA Validation: Precision

Detailed precision information is presented in Table 1.

Analytical (or technical) Variability: A single isolated HDL preparation was injected and analyzed by IMA 6 times during 18 hours (the total analysis time for an entire plate of 72 HDL samples). Each spectrum was processed and deconvoluted in the manner used for the clinical samples (described above). These experiments served two purposes: 1) they established the analytical variability (or technical variability) of calibrated IMA and spectral deconvolution, 2) they demonstrate that HDL samples are stable in the IMA buffer over the time of analysis. The analytical coefficient of variability (CV) was 5.8% for total HDL-P.

Inter-assay Variability: HDLs from 12 plasma samples were isolated and analyzed in triplicate by calibrated IMA. All samples were analyzed in exactly the same manner as the clinical samples. Triplicate isolations and analyses of individual samples were performed in parallel, and the same standard curve was used to calibrate replicates. For total HDL particle concentration, the mean inter-assay CV was 6.2%.

Intra-assay Variability: HDLs from 12 plasma samples were independently isolated and analyzed by calibrated IMA three separate times. All analyses were performed in exactly the same manner as those of the clinical samples. Independent isolations and analyses took place on different days; a unique calibration curve (GOx) was produced for each batch. For total HDL particle concentration, the mean intra-assay CV was 11.4%.

### Calibrated IMA Validation: Robustness

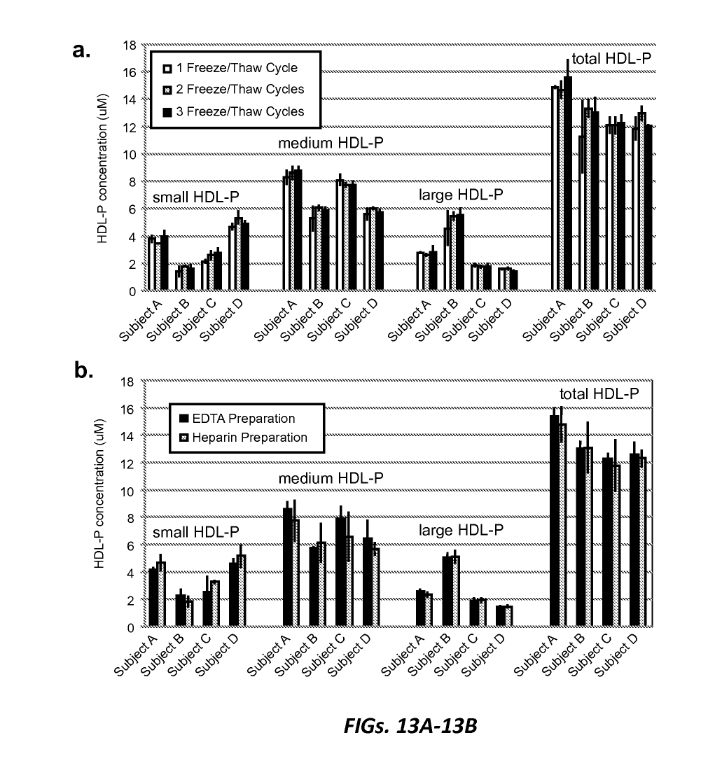

Robustness results are shown in FIGS. 13A-13B.

Freeze-Thaw Effects: Clinical samples are often received as plasma that has been frozen and stored at −80° C. In certain instances, however, frozen plasma samples may be thawed and refrozen more than once. To determine if freeze-thaw cycles affect HDL particle concentrations, aliquots of plasma from four individuals were subjected to one, two, or three rounds of freezing and thawing, and subsequently determined HDL particle concentrations and size by calibrated IMA. Each analysis was performed in triplicate. Particle concentration did not change significantly after one, two, or three freeze/thaw cycles. This stability applied to all three HDL subspecies as well as to total HDL-P. In three plasma samples, the sizes of the HDL subspecies also remained stable. In one plasma sample, the average sm-HDL particle size shifted slightly (0.11 nm) after three freeze/thaw cycles.

Anti-coagulant Effects: Two blood samples were collected in immediate succession from each of 4 study subjects. One set was anticoagulated with EDTA and the other with heparin. Triplicate analyses showed that the type of anticoagulant used had no significant effect on particle concentration for any of the three HDL subspecies or total HDL. Additionally, no differences in HDL subspecies size were observed.

Reconstituted HDL Particles: Reconstituted HDL particles prepared by cholate dialysis were stored at room temperature for 1 week. Calibrated IMA detected no significant changes in particle size or concentration between the reconstituted and freshly prepared particles.

### Statistical Analyses

Statistical tests were performed using R (v2.15.1) or Prism v4.0 (Graphpad). All t-tests were two-tailed and uncorrected. Correlations were evaluated using the method of Pearson. Odds ratios and their confidence intervals were extracted from generalized linear models in R. For all analyses, P values <0.05 were considered significant.

### Discussion

New HDL metrics that provide clinically useful information that persists after adjustment for traditional CVD risk factors—including HDL-C and apoA-I—are urgently needed. HDL-P, the concentration and size of HDL particles in plasma or serum, can represent such a metric. The utility of ion mobility analysis for quantifying HDL-P has been demonstrated herein. The calibrated ion mobility analysis methods described herein can provide an absolute, quantitative measure.

Proteins of different sizes and physiochemical properties yielded linear calibration curves that were essentially superimposable, suggesting that protein standards could be used to quantify other particles of unknown concentration. Consistent with this proposal, the concentrations of reconstituted HDL particles and gold nanoparticles determined by calibrated IMA were in excellent agreement with those determined by orthogonal methods of quantification. Taken together, these observations indicate that calibrated IMA can quantify the concentration of aqueous biological particles in aqueous solution that range widely in size and composition.

Calibrated IMA was next used to investigate the size and concentration of HDL particles in human plasma. Three major subpopulations of HDL particles were independently quantified. The three subspecies of HDL-P closely matched the sizes of a-HDL particles defined by 2D-electrophoresis^{13,26}. Thus, sm-HDL, md-HDL, and lg-HDL likely associate with α3/4-, α2-, and α1-HDL, respectively. IMA spectra of HDL also corresponded well with non-denaturing gradient gel electrophoresis and ultracentrifugal Schlieren patterns, which historically^{27, 28} defined two major HDL subspecies: HDL₂ (p=1.063-1.125 g/mL) corresponding to lg-HDL, and HDL₃ (p=1.125-1.210 g/mL) corresponding to sm-HDL plus md-HDL.

A striking feature of the size distribution data was the marked variability of HDL subspecies profiles. Among individual subjects, for example, the percentage of md-HDL ranged from <15% to >70%; there were similar variations in the fraction of small and large HDL subspecies. In contrast, subspecies diameters were remarkably consistent; each had CVs <3%. These data suggest that genetic and environmental factors can have a major impact on the relative distribution of human HDL subspecies.

A fundamental issue to be resolved is the absolute concentration of HDL particles in blood, which, along with subspecies distribution, is likely to impact HDL's functions. In 7 independent studies, the mean total HDL-P reported by noncalibrated IMA was 5.3 μM, while the average apoA-I concentration was 51 μM (Table 4). These values imply an average stoichiometry of almost 10 apoA-I molecules per HDL particle. In contrast, HDL particle concentrations derived from NMR analyses (n=10) were ˜30 μM (Supp.Table 4), indicating a stoichiometry of ˜1.6 apoA-I molecules per HDL particle. The mean total HDL-P obtained by calibrated IMA was 13.4 μM, with a mean apoA-I value of 48.8 μM, implying 3.6 apoA-I per HDL if all HDL particles contain apoA-I. This stoichiometry is in excellent agreement with abundant biochemical data suggesting an average of 3 to 4 apoA-VHDL and with the current understanding of HDL structure^{14,15}.

TABLE 4
HDL-P by NMR and Non-calibrated Ion Mobility

| | NMR (10 studies) | | Ion Mobility (7 studies) | |
|---|---|---|---|---|
| | n | mean ^{a} | n | mean ^{a} |
| small HDL-P (μM) | 52994 | 20.9 | 9399 | 3.9 |
| medium HDL-P (μM) | 52994 | 4.8 | — ^{b} | — ^{b} |
| large HDL-P (μM) | 52994 | 5.6 | 9399 | 3.4 |
| total HDL-P (μM) | 57100 | 31.4 | 9399 | 5.3 |
| apoA-1 (μM) ^{c} | 4410 | 48.8 | 338 | 50.8 |
| ApoA1/HDL (mole ratio) | — | 1.6 | — | 9.6 |

^{a} Mean of control or pre-treament groups
^{b} IM reports two HDL subspecies; HDL₃₊₂ₐ and HDL_{2b}
^{c} Calculated using ApoA1 molecular weight 28070 Da

In Table 4, HDL-P by NMR:^{S12-21}; HDL-P by IM:^{S22-28}.

The impact of HDL size on sterol efflux was also investigated. Sterol efflux has been proposed to reflect HDL's cardioprotective role. For example, human studies indicate that sterol efflux with J774 macrophages better predicts CVD status than does HDL-C²⁴. Large HDL particles were the most effective mediators of sterol efflux from J774 macrophages, consistent with previous results²⁹. In contrast, smaller, cholesterol-poor HDL particles were the most efficient acceptors of cholesterol from ABCA1. As lipid-free and poorly lipidated apolipoproteins are generally believed to be the major ligands for ABCA1³⁰, these observations suggest that small HDL may play a role in reverse cholesterol transport by the ABCA1 pathway. They also demonstrate that calibrated IMA can provide important insights into HDL function.

Because HDL subspecies have been linked to CVD risk¹³ and show differential function as well as composition, a key question is whether HDL-P is a better metric of CVD risk than HDL-C. It was found that HDL-P associated strongly and inversely with carotid cerebral vascular disease and that decreased levels of md-HDL particles accounted largely for that association. Importantly, differences in total HDL-P and md-HDL-P remained significant after adjustment for HDL-C, suggesting that HDL-P can be distinct from HDL-C. Indeed, HDL-C predicted only 50% of total HDL-P variance, and evidence is provided that variable subspecies distribution was a key mechanism dissociating the two HDL metrics. The association of low HDL-P with CCVD persisted after adjustment for other vascular risk factors, including LDL-C, triglycerides, age, and sex.

In conclusion, a method for determining the size and absolute concentration of HDL in human blood is described. HDL-P yielded a value for the stoichiometry of apoA-I per HDL particle that fit well with the current understanding of HDL structure. It was also the strongest predictor of CCVD status in a clinical population. The association of low HDL-P with carotid cerebral vascular disease was independent of HDL-C, apoA-I, and traditional CVD risk factors. These observations indicate that quantifying HDL particle concentration and size can provide more clinically relevant information about HDL's cardioprotective functions than measuring HDL-C levels. That is, calibrated IMA methods described herein can provide more relevant diagnostic information than existing approaches to assess CVD risk.

### Endothelial Dysfunction

HDL-P independently associates with endothelial dysfunction (ED). Early atherosclerosis of the coronary arteries may be associated with regional inflammation and increased blood levels of inflammatory markers. Early atherosclerosis strongly associates with ED, which is caused by an imbalance between endothelium-dependent vasodilator and vasoconstrictor activity, as well as by inflammation and other factors (Lavi S, McConnell J P, Rihal C S, Prasad A, Mathew V, Lerman L O, Lerman A. Local production of lipoprotein-associated phospholipase A2 and lysophosphatidylcholinen the coronary circulation: association with early coronary atherosclerosis and endothelial dysfunction in humans. Circulation. 2007 May 29; 115(21):2715-21).

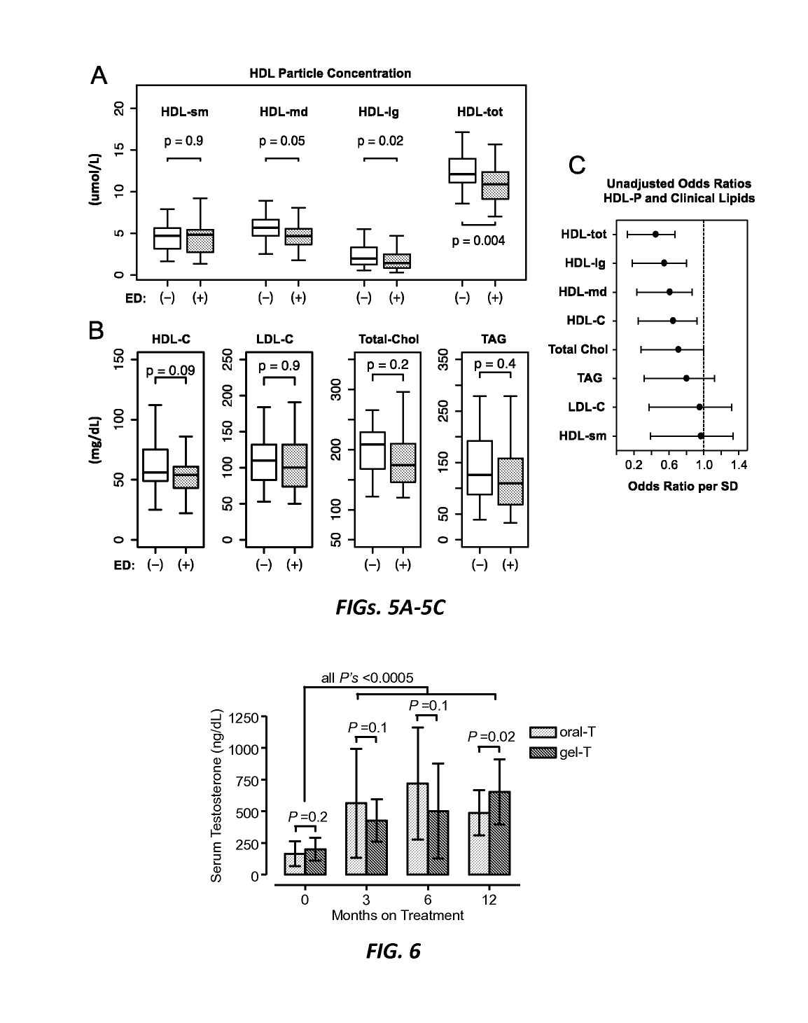

To explore whether calibrated IMA can be a clinically useful alternative to HDL-C in ED subjects, defined as vasoconstriction of their coronary vasculature when challenged with acetylcholine, plasma samples of 34 of the patients positive for ED and 38 patients that had a normal response to acetylcholine were studied (FIG. 5). Subjects with ED had 13% lower total HDL-P than controls (10.96 vs 12.64; p=0.004), 37% lower lg-HDL (1.68 vs 2.52 μmol/L, p=0.02), 14% lower md-HDL (4.82 vs 5.61 μmol/L; p=0.05) and essentially equivalent sm-HDL (4.51 vs 4.46 μmol/L; p=0.9) (FIG. 5A). None of the classic lipid risk factors, e.g. HDL-C, LDL-C, total cholesterol, or triglycerides, showed a significant differences between the groups, although HDL-C was trending lower in subjects ED vs controls (53.2 vs 60.9 mg/dL) (FIG. 5B).

To assess the independent predictive value of HDL-P with respect to HDL-C, generalized linear models were constructed including both variables. After adjustment for HDL-C, differences in total HDL-P remained significant (p=0.03) although differences in md-HDL and lg-HDL were no longer significant. When unadjusted odds ratios were calculated (FIG. 5C) total HDL-P, followed by lg-HDL and md-HDL, all were significant negative predictors of ED risk. HDL-C was also a significant negative predictor, however, not as strong. LDL-C, total cholesterol, triglycerides and sm-HDL were not significant predictors. When logistic regression models included age and sex, the strength and significance of the odds ratios was identical.

These observations indicate that total HDL-P and large HDL-P were significantly associated with ED status in these subjects and were both better predictors of ED status than HDL-C. Importantly, differences in total HDL-P persisted after adjusting for HDL-C. Comparing these data to the CLEAR study (where medium HDL-P was most predictive), these observations indicate that different HDL subspecies can be altered in different clinical populations with established CVD. They also strongly indicate that HDL-P is a much better predictor of ED status than HDL-C, indicating that calibrated IMA can provide unique insights into CVD risk, and even HDL-targeted therapeutics, in ED subjects.

### Testosterone Therapy

HDL-P associates with testosterone therapy in hypogonadal males. Testosterone levels decline in men as they age, and this strongly associates with changes in BMI and insulin resistance, known cardiac risk factors. Short-term studies indicate that testosterone lowers HDL-C levels, but it is not yet clear if long-term therapy with testosterone in men associates with increased or decreased CVD risk (Ruige J B, Ouwens D M, Kaufman J M. Beneficial and adverse effects of testosterone on the cardiovascular system in men. J Clin Endocrinol Metab. 2013 November; 98(11):4300-10). Nor has the impact of testosterone therapy on CVD risk in hypogonadal men been established.

To determine whether HDL-P might provide information on CVD risk, the impact of testosterone therapy on HDL-C and HDL-P in hypogonadal men was studied.

Patient Population.

Hypogonadal male subjects (n=54) undergoing testosterone replacement therapy were randomized to one of two formulations; either transdermal gel testosterone (gel-T, n=27) or oral testosterone (oral-T, n=27). Blood samples were collected at baseline (day 0), and after approximately three, six and twelve months on treatment. HDL particle concentration (HDL-P), HDL cholesterol (HDL-C) and testosterone levels were determined for each subject at each time point.

Statistical Analysis.

Statistical tests were performed using R (v2.15.1). Comparisons between baseline (time=0) and on-treatment values (times>0) were performed by paired Student's t-tests. Comparisons between groups at a given time point (e.g. oral-T vs. gel-T at 12 months) were performed by independent t-tests. All t-tests were two-tailed and uncorrected. P-values below 0.05 were considered significant.

### Results and Discussion

Testosterone replacement therapy has been associated with increased risk of cardiovascular disease (CVD) related events. Because testosterone treatment significantly depresses HDL-C levels, but does not alter other lipid risk factors, such LDL-C or triacylglyceride (triglycerides), HDL is implicated as a causal factor. To further investigate the effects of testosterone, the concentration of HDL particles was measured in hypogonadal males undergoing hormone replacement therapy with two different drug formulations: an oral form and a transdermal form. The results revealed highly differential effects of the two treatments. They also demonstrated that HDL particle concentration and HDL-C measures are distinct and provide unique information.

Oral and Gel Formulations Achieved Similar Testosterone Levels:

Both formulations significantly raised testosterone levels above baseline at all time points (FIG. 6). In the short-term, oral-T trended higher but both groups had statistically equivalent testosterone levels after 3 and 6 months on-treatment. At 12 months, subjects receiving gel-T had significantly higher testosterone levels.

Oral-T Decreases Large HDL Particles and HDL-C:

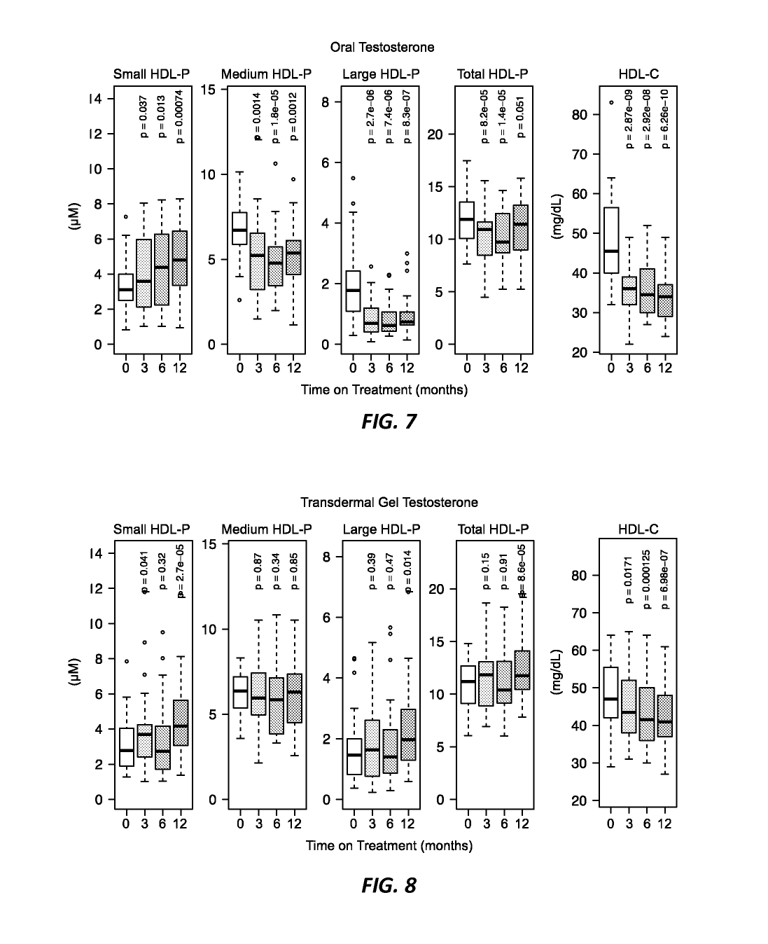

The HDL-C lowering effect of testosterone replacement therapy was especially apparent in subjects receiving oral-T. In this group HDL-C was decreased 27% after three months of treatment (FIG. 7). This was likely due to a striking decline (by >50%) in the concentration of large, cholesterol-rich HDL particles. In contrast, gel-T subjects showed only an 8% decline in HDL-C (P=0.02) at three months and no change in lg-HDL-P (FIG. 8). Since both treatment groups had similar circulating testosterone levels at three months (P=0.1) these data most likely reflect pharmacokinetic differences between the two formulations. The observation that the oral formulation caused a more pronounced effect is consistent with the hypothesis that increased lipase activity, driven by testosterone in the liver, initially acted to degrade large lipid-rich HDLs.

Long-Term Conservation of Total HDL-P in Oral-T Subjects:

At early time points, HDL-C levels reflected changes in large particle concentration, however; other effects of testosterone replacement therapy were only apparent in HDL-P. Most strikingly, in oral-T subjects total HDL-P returned to baseline levels at twelve months (P>0.05), while HDL-C and lg-HDL-P remained significantly depressed (P's<0.001) (FIG. 7). A steady increase in small HDL particle concentration, over the entire study, effectively equilibrated the total number of circulating HDL particles. The overall result was that oral-T subjects have clearly altered HDL (smaller HDL) but the same number total particles. These observations demonstrate that HDL-P provides unique information independent of HDL-C and suggests that reproducible quantitation of HDL subpopulations can provide new insights into the nature of individual subspecies.

HDL-P Implicates Alterations of HDL Lipids in Gel-T Subjects:

At twelve months HDL-C levels in the gel-T subjects were moderately, but significantly, decreased by 13% (P<0.001) (FIG. 8). Interestingly, there was no drop in the concentration of large or medium sized HDL particles, in fact, lg-HDL-P was slightly increased at 12 months (P=0.01). These changes in gel-T subjects were relatively small, and random variation might provide a trivial explanation for these observations. Another possibility is that HDL cholesterol was exchanged for triglycerides, thereby lowering HDL-C without significant changes to lg- or md-HDL-P. Two lines of evidence support this hypothesis: 1) sm-HDL-P, which is positively associated with triglycerides and negatively associated with HDL-C, is significantly increased at 12 months (30%, P<0.001) and 2) alterations in lipase activity, such as in hepatic lipase deficiency, often result in triglycerides enriched HDLs. Considering the likely involvement of lipolytic enzymes in oral-T-induced HDL alterations, changes to the lipid cargo of HDL in gel-T subjects may explain these observations. Studies combining HDL-P measures with lipidomics may shed new light on hormonal regulation of plasma lipids.

In subjects receiving oral-T, decreases in HDL-C where likely due to degradation of large HDL particles. Because this effect was pronounced in the oral formulation, high testosterone concentrations in the liver were implicated. In oral-T subjects total HDL-P returned baseline by 12 months, this phenomenon was not captured by HDL-C. In gel-T subjects, decreases in HDL-C, without degradation of large or medium HDL particles, suggested alterations in HDL lipid cargo. These data further suggest that HDL particle concentration is unique from HDL-C and may serve a useful purpose in drug development.

### Chronic Kidney Disease

HDL-P associates with chronic kidney disease. Chronic kidney disease is a major risk factor for accelerated atherosclerosis and greatly increased CVD risk (Go A S, Chertow G M, Fan D, McCulloch C E, Hsu C Y. Chronic kidney disease and the risks of death, cardiovascular events, and hospitalization. N Engl J Med. 2004 Sep. 23; 351(13):1296305). However, the underlying mechanisms remain poorly understood, traditional lipid risk factors (LDL-C and HDL-C) do not appear to be strongly linked to CVD risk, and conventional therapies directed towards lowering LDL-C levels appear to less effective at lowering CVD risk than in subjects with normal kidney function (Fellstrom B C, Jardine A G, Schmieder R E, Holdaas H, Bannister K, Beutler J, Chae D W, Chevaile A, Cobbe S M, Gronhagen-Riska C, De Lima J J, Lins R, Mayer G, McMahon A W, Parving H H, Remuzzi G, Samuelsson 0, Sonkodi S, Sci D, Silleymanlar G, Tsakiris D, Tesar V, Todorov V, Wiecek A, Wiithrich R P, Gottlow M, Johnsson be E, Zannad F; AURORA Study Group. Rosuvastatin and cardiovascular events in patients undergoing hemodialysis. N Engl J Med. 2009 Apr. 2; 360(14):1395-407. Baigent C, Landray M J, Reith C, Emberson J, Wheeler D C, Tomson C, Wanner C, Krane V, Cass A, Craig J, Neal B, Jiang L, Hooi L S, Levin A, Agodoa L, Gaziano M, Kasiske B, Walker R, Massy Z A, Feldt-Rasmussen B, Krairittichai U, Ophascharoensuk V, Fellstrom B, Holdaas H, Tesar V, Wiecek A, Grobbee D, de Zeeuw D, Gronhagen-Riska C, Dasgupta T, Lewis D, Herrington W, Mafham M, Majoni W, Wallendszus K, Grimm R, Pedersen T, Tobert J, Armitage J, Baxter A, Bray C, Chen Y, Chen Z, Hill M, Knott C, Parish S, Simpson D, Sleight P, Young A, Collins R; SHARP Investigators. The effects of lowering LDL cholesterol with simvastatin plus ezetimibe in patients with chronic kidney disease (Study of Heart and Renal Protection): a randomized placebo-controlled trial. Lancet. 2011 Jun. 25; 377(9784):2181-92).

Moreover, CKD strongly associates with increased inflammation (Oberg B P, McMenamin E, Lucas F L, McMonagle E, Morrow J, Ikizler T A, Himmelfarb J. Increased prevalence of oxidant stress and inflammation in patients with moderate to severe chronic kidney disease. Kidney Int. 2004 March; 65(3):1009-16). And many lines of evidence indicate that HDL can inhibit inflammation in animal models, raising the possibility that HDL-targeted therapies might lower CVD risk in CKD subjects.

To explore whether calibrated IMA can be a clinically useful alternative to HDL-C in chronic kidney disease (CKD) subjects, plasma samples of 40 patients on dialysis and 20 control patients that had normal renal function were studied (Table 1).

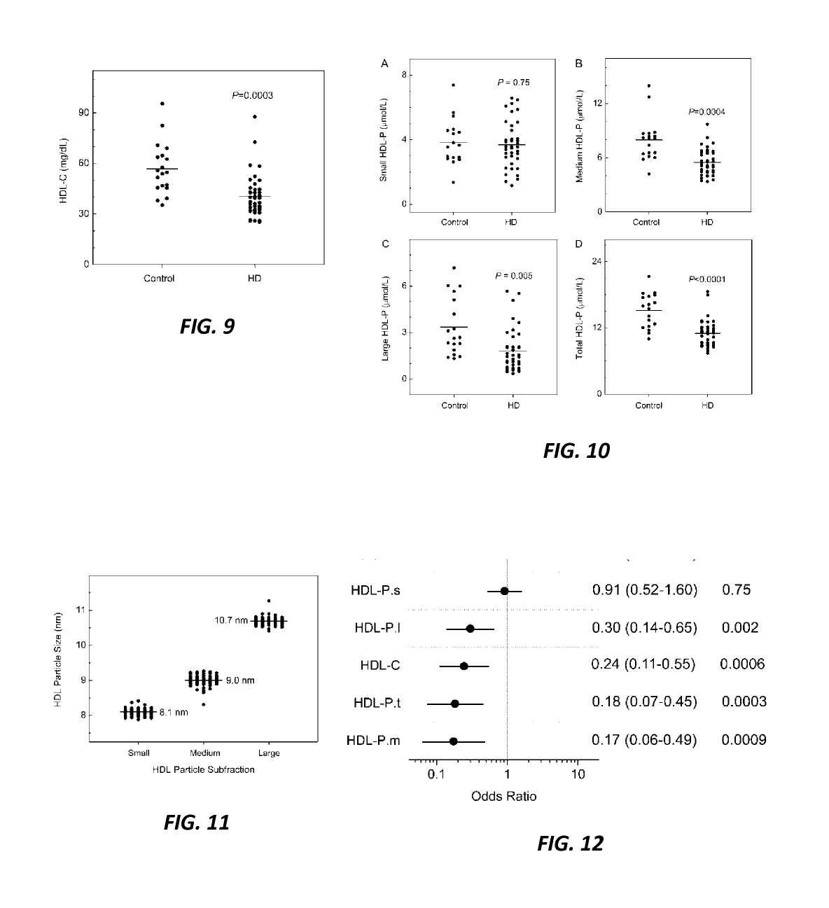

Significant decreases were found in HDL-C (P=0.0003) and HDL-P (P=0.0001) in the subjects with CKD than in the control subjects (FIG. 9 and FIG. 10). As in control subjects, CVD subjects, and hypogonadal male subjects, there were three major sizes of HDL particles (FIG. 11). The concentrations of total HDL-P, md-HDL-P and lg-HDL-P, but not sm-HDL-P, were significantly lower in the CKD subjects than the control subjects.

The ORs and confidence intervals were calculated for the association of HDL-C and HDL-P with CKD status (FIG. 12). Low levels of md-HDL-P and HDL-P strongly associated with CKD status. Low levels of HDL-C and lg-HDL-P were less strongly associated with CKD status, and sm-HDL-P was not associated with CKD status.

These observations indicate that low levels of HDL-P, lg-HDL-P and md-HDL-P were significantly associated with CKD status in these subjects. Both HDL-P and md-HDL-P were more strongly associated with CKD status than was HDL-C. These observations again indicate that in this population the specific subspecies of HDL particles were affected. They also strongly suggest that HDL-P is a better predictor of CKD status than HDL-C, indicating that calibrated IMA can provide unique insights into CKD risk, and HDL-targeted therapeutics, in CKD subjects.

### REFERENCES FOR EXAMPLE 1

1. Ordovas, J. M. et al. Apolipoprotein A-I gene polymorphism associated with premature coronary artery disease and familial hypoalphalipoproteinemia. N. Engl. J. Med. 314, 671-677 (1986).
2. Moore, R. E. et al. Increased atherosclerosis in mice lacking apolipoprotein A-I attributable to both impaired reverse cholesterol transport and increased inflammation. Circ. Res. 97, 763-771 (2005).
3. Attie, A. D., Kastelein, J. P. & Hayden, M. R. Pivotal role of ABCA1 in reverse cholesterol transport influencing HDL levels and susceptibility to atherosclerosis. J. Lipid Res. 42, 1717-1726 (2001).
4. Gordon, D. J. & Rifkind, B. M. High-density lipoprotein—the clinical implications of recent studies. N. Engl. J. Med. 321, 1311-1316 (1989).
5. Rader, D. J. & Tall, A. R. The not-so-simple HDL story: Is it time to revise the HDL cholesterol hypothesis? Nat. Med. 18, 1344-1346 (2012).
6. Barter, P. J. et al. Antiinflammatory properties of HDL. Circ. Res. 95, 764-772 (2004).
7. Voight, B. F. et al. Plasma HDL cholesterol and risk of myocardial infarction: a mendelian randomisation study. Lancet 380, 572-580 (2012).
8. AIM-HIGH Investigators et al. Niacin in patients with low HDL cholesterol levels receiving intensive statin therapy. N. Engl. J. Med. 365, 2255-2267 (2011).
9. Schwartz, G. G. et al. Effects of dalcetrapib in patients with a recent acute coronary syndrome. N. Engl. J. Med. 367, 2089-2099 (2012).
10. Warden, C. H., Hedrick, C. C., Qiao, J. H., Castellani, L. W. & Lusis, A. J. Atherosclerosis in transgenic mice overexpressing apolipoprotein A-II. Science 261, 469-472 (1993).
11. Braun, A. et al. Loss of SR-BI expression leads to the early onset of occlusive atherosclerotic coronary artery disease, spontaneous myocardial infarctions, severe cardiac dysfunction, and premature death in apolipoprotein E-deficient mice. Circ. Res. 90, 270-276 (2002).
12. Vergeer, M., Holleboom, A. G., Kastelein, J. J. P. & Kuivenhoven, J. A. The HDL hypothesis: does high-density lipoprotein protect from atherosclerosis? J. Lipid Res. 51, 2058-2073 (2010).
13. Asztalos, B. F., Tani, M. & Schaefer, E. J. Metabolic and functional relevance of HDL subspecies. Curr. Opin. Lipidol. 22, 176-185 (2011).
14. Shen, B. W., Scanu, A. M. & Kezdy, F. J. Structure of human serum lipoproteins inferred from compositional analysis. Proceedings of the National Academy of Sciences 74, 837-841 (1977).
15. Huang, R. et al. Apolipoprotein AI structural organization in high-density lipoproteins isolated from human plasma. Nature structural & molecular biology 18, 416-422 (2011).
16. Jeyarajah, E. J., Cromwell, W. C. & Otvos, J. D. Lipoprotein particle analysis by nuclear magnetic resonance spectroscopy. Clin. Lab. Med. 26, 847-870 (2006).
17. Caulfield, M. P. et al. Direct determination of lipoprotein particle sizes and concentrations by ion mobility analysis. Clin. Chem. 54, 1307-1316 (2008).
18. Mackey, R. H. et al. High-Density Lipoprotein Cholesterol and Particle Concentrations, Carotid Atherosclerosis, and Coronary Events: MESA (Multi-Ethnic Study of Atherosclerosis). Journal of the American College of Cardiology doi:10.1016/j.jacc.2012.03.060
19. Mora, S., Glynn, R. J. & Ridker, P. M. High-density lipoprotein cholesterol, size, particle number, and residual vascular risk after potent statin therapy. Circulation 128, 1189-1197 (2013).
20. Guha, S., Li, M., Tarlov, M. J. & Zachariah, M. R. Electrospray—differential mobility analysis of bionanoparticles. Trends in biotechnology (2012). at <http://www.sciencedirect.com/science/article/pii/S0167779912000182>
21. Page, J. S., Kelly, R. T., Tang, K. & Smith, R. D. Ionization and Transmission Efficiency in an Electrospray Ionization—Mass Spectrometry Interface. Journal of the American Society for Mass Spectrometry 18, 1582-1590 (2007).
22. Swaney, J. B. Properties of lipid-apolipoprotein association products. Complexes of human apo AI and binary phospholipid mixtures. J. Biol. Chem. 255, 8798-8803 (1980).
23. Cavigiolio, G. et al. The interplay between size, morphology, stability, and functionality of high-density lipoprotein subclasses. Biochemistry 47, 4770-4779 (2008).
24. Khera, A. V. et al. Cholesterol efflux capacity, high-density lipoprotein function, and atherosclerosis. N. Engl. J. Med. 364, 127-135 (2011).
25. Jarvik, G. P. et al. Paraoxonase (PON1) phenotype is a better predictor of vascular disease than is PON1(192) or PON1(55) genotype. Arterioscler. Thromb. Vasc. Biol. 20, 2441-2447 (2000).
26. Asztalos, B. F., Sloop, C. H., Wong, L. & Roheim, P. S. Two-dimensional electrophoresis of plasma lipoproteins: recognition of new apo A-I-containing subpopulations. Biochim. Biophys. Acta 1169, 291-300 (1993).
27. Rosenson, R. S. et al. HDL measures, particle heterogeneity, proposed nomenclature, and relation to atherosclerotic cardiovascular events. Clin. Chem. 57, 392-410 (2011).
28. DELALLA, 0. F., ELLIOTT, H. A. & GOFMAN, J. W. Ultracentrifugal studies of high density serum lipoproteins in clinically healthy adults. Am. J. Physiol. 179, 333-337 (1954).
29. Matsuura, F., Wang, N., Chen, W., Jiang, X.-C. & Tall, A. R. HDL from CETP-deficient subjects shows enhanced ability to promote cholesterol efflux from macrophages in an apoE- and ABCG1-dependent pathway. J. Clin. Invest. 116, 1435-1442 (2006).
30. Oram, J. F. & Heinecke, J. W. ATP-binding cassette transporter A1: a cell cholesterol exporter that protects against cardiovascular disease. Physiol. Rev. 85, 1343-1372 (2005).
31. Vaisar, T. et al. Shotgun proteomics implicates protease inhibition and complement activation in the antiinflammatory properties of HDL. J. Clin. Invest. 117, 746-756 (2007).
32. Shao, B., Tang, C., Heinecke, J. W. & Oram, J. F. Oxidation of apolipoprotein A-I by myeloperoxidase impairs the initial interactions with ABCA1 required for signaling and cholesterol export. J. Lipid Res. 51, 1849-1858 (2010).
33. Hoppel, W. A. Determination of the aerosol size distribution from the mobility distribution of the charged fraction of aerosols. Journal of Aerosol Science 9, 41-54 (1978).
34. Wojdyr, M. Fityk: a general-purpose peak fitting program. Journal of Applied Crystallography 43, 1126-1128 (2010).
S1. Kaddis, C. S. & Loo, J. A. Native protein MS and ion mobility: Large flying proteins with ESI. Anal. Chem. 79, 1778-1784 (2007).
S2. Guha, S., Li, M., Tarlov, M. J. & Zachariah, M. R. Electrospray—differential mobility analysis of bionanoparticles. Trends Biotechnol. (2012). at <http://www.sciencedirect.com/science/article/pii/S0167779912000182>
S3. Kapellios, E. A. et al. Using nanoelectrospray ion mobility spectrometry (GEMMA) to determine the size and relative molecular mass of proteins and protein assemblies: a comparison with MALLS and QELS. Anal. Bioanal. Chem. 399, 2421-2433 (2011).
S4. Kaddis, C. S. et al. Sizing large proteins and protein complexes by electrospray ionization mass spectrometry and ion mobility. J. Am. Soc. Mass Spectrom. 18, 1206-1216 (2007).
S5. Bacher, G. et al. Charge-reduced nano electrospray ionization combined with differential mobility analysis of peptides, proteins, glycoproteins, noncovalent protein complexes and viruses. J. Mass Spectrom. JMS 36, 1038-1052 (2001).
S6. Marty, M. T. et al. Native Mass Spectrometry Characterization of Intact Nanodisc Lipoprotein Complexes. Anal. Chem. 84, 8957-8960 (2012).
S7. Bayburt, T. H. & Sligar, S. G. Membrane protein assembly into Nanodiscs. FEBS Lett. 584, 1721-1727 (2010).
S8. Cavigiolio, G. et al. The interplay between size, morphology, stability, and functionality of high-density lipoprotein subclasses. Biochemistry (Mosc.) 47, 4770-4779 (2008).
S9. Scanu, A., Reader, W. & Edelstein, C. Molecular weight and subunit structure of human serum high density lipoprotein after chemical modification by succinic anhydride. Biochim. Biophys. Acta BBA—Protein Struct. 160, 32-45 (1968).
S10. Flagan, R. C. Differential mobility analysis of aerosols: a tutorial. KONA Powder Part. J. 254-258 (2008).
S11. Fuchs, N. A. On the stationary charge distribution on aerosol particles in a bipolar ionic atmosphere. Geofis. Pura E Appl. 56, 185-193 (1963).
S12. Mora, S. et al. LDL particle subclasses, LDL particle size, and carotid atherosclerosis in the Multi-Ethnic Study of Atherosclerosis (MESA). Atherosclerosis 192, 211-217 (2007).
S13. Mora, S. et al. Lipoprotein particle size and concentration by nuclear magnetic resonance and incident type 2 diabetes in women. Diabetes 59, 1153-1160 (2010).
S14. Jeyarajah, E. J., Cromwell, W. C. & Otvos, J. D. Lipoprotein particle analysis by nuclear magnetic resonance spectroscopy. Clin. Lab. Med. 26, 847-870 (2006).
S15. Mackey, R. H. et al. High-Density Lipoprotein Cholesterol and Particle Concentrations, Carotid Atherosclerosis, and Coronary Events: MESA (Multi-Ethnic Study of Atherosclerosis). J. Am. Coll. Cardiol. doi:10.1016/j.jacc.2012.03.060
S16. Rosenson, R. S., Otvos, J. D. & Hsia, J. Effects of rosuvastatin and atorvastatin on LDL and HDL particle concentrations in patients with metabolic syndrome: a randomized, double-blind, controlled study. Diabetes Care 32, 1087-1091 (2009).
S17. Hsia, J. et al. Lipoprotein particle concentrations may explain the absence of coronary protection in the women's health initiative hormone trials. Arterioscler. Thromb. Vasc. Biol. 28, 1666-1671 (2008).
S18. El Harchaoui, K. et al. High-density lipoprotein particle size and concentration and coronary risk. Ann. Intern. Med. 150, 84-93 (2009).
S19. Otvos, J. D. et al. Low-density lipoprotein and high-density lipoprotein particle subclasses predict coronary events and are favorably changed by gemfibrozil therapy in the Veterans Affairs High-Density Lipoprotein Intervention Trial. Circulation 113, 1556-1563 (2006).
S20. Berger, J. S. et al. Lipid and lipoprotein biomarkers and the risk of ischemic stroke in postmenopausal women. Stroke J. Cereb. Circ. 43, 958-966 (2012).
S21. Virani, S. S. et al. Relation of cholesterol and lipoprotein parameters with carotid artery plaque characteristics: the Atherosclerosis Risk in Communities (ARIC) carotid MRI study. Atherosclerosis 219, 596-602 (2011).
S22. Caulfield, M. P. et al. Direct determination of lipoprotein particle sizes and concentrations by ion mobility analysis. Clin. Chem. 54, 1307-1316 (2008).
S23. Musunuru, K. et al. Ion mobility analysis of lipoprotein subfractions identifies three independent axes of cardiovascular risk. Arterioscler. Thromb. Vasc. Biol. 29, 1975-1980 (2009).
S24. Page, S. T. et al. Impact of mifepristone, a glucocorticoid/progesterone antagonist, on HDL cholesterol, HDL particle concentration, and HDL function. J. Clin. Endocrinol. Metab. 97, 1598-1605 (2012).
S25. Sonestedt, E. et al. High disaccharide intake associates with atherogenic lipoprotein profile. Br. J. Nutr. 107, 1062-1069 (2012).
S26. Krauss, R. M. et al. Changes in lipoprotein subfraction concentration and composition in healthy individuals treated with the CETP inhibitor anacetrapib. J. Lipid Res. 53, 540-547 (2012).
S27. Mietus-Snyder, M. L. et al. A nutrient-dense, high-fiber, fruit-based supplement bar increases HDL cholesterol, particularly large HDL, lowers homocysteine, and raises glutathione in a 2-wk trial. FASEB J. Off. Publ. Fed. Am. Soc. Exp. Biol. 26, 3515-3527 (2012).
S28. Choi, Y.-J. et al. Effects of the PPAR-δ agonist MBX-8025 on atherogenic dyslipidemia. Atherosclerosis 220, 470-476 (2012).

### Example 2

It should be noted that two classification schemes for HDL subspecies have been proposed, based on apparent size (VS-HDL, S-HDL, M-HDL, L-HDL: very small, small, medium, and large HDL) and migration on 2D-GE (α-4 to α-1). See Table 5, below, for a key mapping the two sets of definitions to the other.

TABLE 5
Classification of HDL particles based on size
Nomenclature

| Size | 2D GE |
|---|---|
| VS-HDL | α-4 |
| S-HDL | α-3 |
| M-HDL | α-2 |
| L-HDL | α-1 |

Quanting HDL Particle Number and Size by Calibrated Ion Mobility Analysis (IMA).

One potentially useful metric is HDL-P (the concentration of HDL particles), as HDL is a complex mixture of particles that range in size from 7 nm to 12 nm and vary 4-fold in cholesterol content.

The ion mobility-based method for HDL-P measurement has been extended by implementing internal standards (Hutchins P. et al., Quantification of HDL Particle Concentration by Calibrated Ion Mobility Analysis. Clin Chem. 2014 Sep. 15). Importantly, it was extensively calibrated and validated to establish the method termed calibrated IMA. By using particles of known size, shape, and concentration for calibration, an average value for human HDL-P of ˜15 μM was obtained, with average precision <10% CV. It has also been confirmed that the method described herein yields a correct value for the concentration of synthetic HDL particles and gold nanoparticles and their molecular size. Moreover, HDL-C does not directly quantify either HDL-P or HDL size. The relationship between HDL-C and HDL-P is weak (r²˜0.3).

In contrast to the previously reported concentration by ion mobility, ˜4-5 μM, or NMR, 35 μM, the values obtained herein for HDL-P (˜15 μM) and particle size (using clinical samples) are in excellent agreement with the values predicted from the composition of isolated HDL (3-4 apoA-I molecules per spherical HDL particle). They also are in excellent agreement with the current understanding of HDL structure.

HDLs in LCAT-deficient subjects are homogenous in size but vary in concentration. HDL was isolated from three healthy control subjects and three subjects with homozygous LCAT deficiency. The HDLs from the controls showed a highly heterogeneous size distribution. Major peaks corresponding to all of the three major HDL subspecies were apparent, and the mean total HDL particle concentration (HDL-P) was near 17 μM—typical for healthy subjects. HDLs of the LCAT-deficient subjects gave a dramatically different result, because a single abundant subspecies, corresponding in size to α-4 (very small) HDL, was observed. Two LCAT-deficient subjects (FIG. 16, subjects A,B) had total HDL-P near 6 μM (V3 normal), while one (FIG. 16, subject C) had substantially lower HDL-P—near 3 μM. Furthermore, two subjects had extremely homogenous α-4 HDLs (FIG. 16, subjects A,C), while one had a skewed α-4 peak (FIG. 16, subject B) that likely contained some α-3 HDL and a minor subspecies >9 nm in diameter corresponding to α-2 HDL. These data show that HDLs in LCAT-deficient subjects are not equivalent and suggest that different LCAT mutations have differential effects on HDL. Significantly, these initial analyses provide evidence that LCAT-deficient subjects have normal or higher than normal concentrations of α-4 (very small) HDL particles (FIG. 16, subjects A,B). It is interesting to note that people with Tangier's disease, who have normal or high pre-β HDL but no α-4 HDL, clearly suffer from early onset CVD while LCAT-deficient subjects do not. This observation suggests that α-4 HDL and/or its production can have an important impact on vascular cholesterol trafficking.

In view of the above experimental evidence, HDL-P of certain subspecies in LCAT-deficient subjects reflects important aspects of HDL-mediated cardioprotection.

Subspecies and total HDL particle concentrations of LCAT-deficient and control subjects are measured and analyzed according to methods of the present application.

New HDL metrics have thereby been identified that better reflect the cardioprotective effects of HDL than does HDL-C, the current gold standard. Specifically, higher concentrations of α4/vs-HDL particles are indicative of a lower risk of CVD. Thus, HDL-P can help clinical lipidologists identify and characterize HDL particles that are indicative of CVD presence or risk and also inform treatment recommendations for specific patients.

### Example 3

HDL was isolated from plasma by ultracentrifugation, introduced into the gas phase with electrospray ionization, separated by size, and quantified by particle counting. A calibration curve constructed with purified proteins was used to correct for the ionization efficiency of HDL particles.

The concentrations of gold nanoparticles and reconstituted HDLs measured by calibrated IMA were indistinguishable from concentrations determined by orthogonal methods. In plasma of control (n=40) and cerebrovascular disease (n=40) subjects, three subspecies of HDL were reproducibility measured, with an estimated total HDL-P of 13.4±2.4 μM (mean±SD). HDL-C accounted for 48% of the variance in HDL-P. HDL-P was significantly lower in subjects with cerebrovascular disease (P=0.002), and this difference remained significant after adjustment for HDL cholesterol concentrations (P=0.02).

Calibrated IMA accurately determined the concentration of gold nanoparticles and synthetic HDL, strongly suggesting the method could accurately quantify HDL particle concentration. The estimated stoichiometry of apoA-I determined by calibrated IMA was 3-4 per HDL particle, in agreement with current structural models. Furthermore, HDL-P associated with cardiovascular disease status in a clinical population independently of HDL cholesterol.

### Materials and Methods

HDL Preparation.

Total lipoproteins were isolated from plasma in a single ultracentrifugation step as follows: 50 μL plasma, 50 μL normal saline (with 0.5 mM EDTA), and 130 μL KBr (p=1.37 g/mL) were added to 7×20 mm ultracentrifugation tubes (final p=1.21 g/mL). Tubes were centrifuged in a 72-position rotor (type 42.2 TI) at 42,000 rpm (214, 361×g, average) for 12 h; 57 μL was then taken from the top of each tube and placed in a 96-well constant-flow dialyzer (Spectrum Laboratories Inc.). Samples were dialyzed for 4 h at 4° C. against NH₄OAc (5 mM, adjusted to pH 7.4 with NH₄OH) at a flow-rate of ˜5 mL/min. Immediately prior to analysis, samples were diluted 500-fold (relative to the original plasma volume) with NH₄OAc (5 mM, pH 9.2).

Human transferrin (T8158), bovine catalase (C40), Aspergillus niger glucose oxidase (G2133), cholesterol and sodium deoxycholate were obtained from Sigma-Aldrich. Ultrapure human apoA-I was purchased from Academy Biomedical Co. Palmitoyl-oleoyl-phosphatidylcholine was obtained from Avanti Polar Lipids (Alabaster, Ala.). Ammonium acetate, A.C.S. grade (NH₄OAc), and ammonium hydroxide, A.C.S. plus grade (NH₄OH), were obtained from Fisher Scientific. Polyvinylpyrrolidone coated gold nanoparticles (10 nm; NanoXact) were purchased from nanoComposix.

Recovery of HDL by Ultracentrifugation.

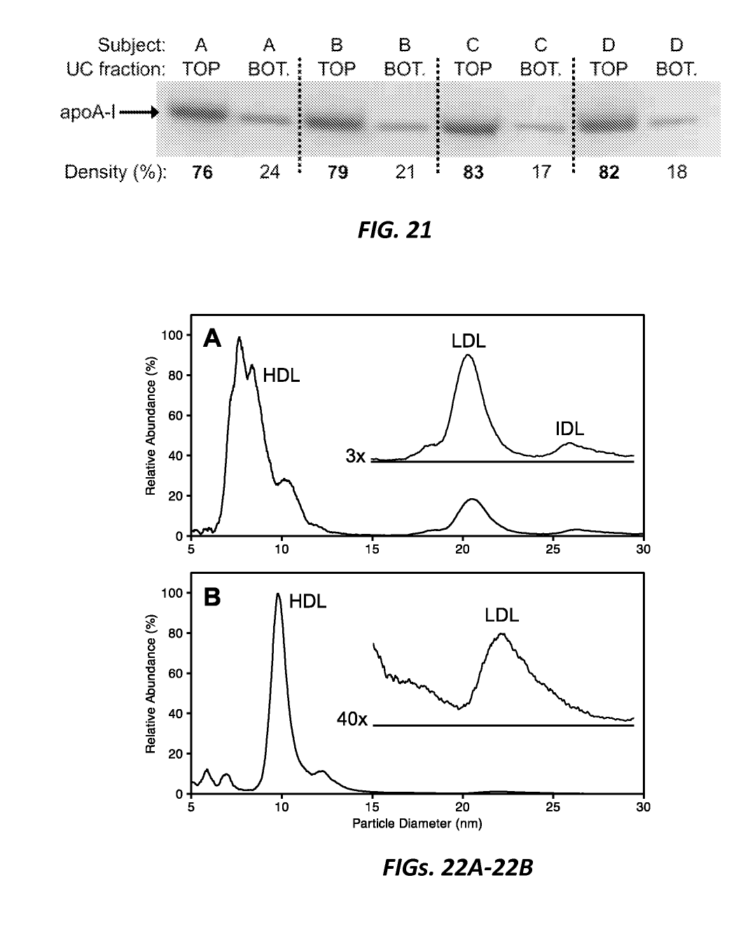

To estimate HDL recovery from plasma by ultracentrifugation, the apoA-I content of the HDL and non-HDL fractions was quantified. HDLs from the plasma of 4 individuals were isolated as described somewhere else in this application. Equal proportions of the top and bottom ultracentrifuge fractions (corresponding to 2 μL of plasma; both fractions were homogenized to avoid sampling error) were separated by SDS-PAGE and immunoblotted with a polyclonal antibody to apoA-I (Meridian Life Sciences). Immunoreactive protein was quantified by the “rolling ball” method (Gassmann M, et al., ELECTROPHORESIS. 2009; 30:1845-55). The estimated apoA-I recovery in the HDL fraction was 80±3% (mean±SD; FIG. 21).

Differential Ion Mobility.

Briefly, analytes in aqueous solution are converted to gas-phase ions by ESI (FIGS. 15A-15C). The resulting highly charged ions are largely neutralized by alpha particles, yielding a small proportion of singly charged cations, which are introduced into the mobility analyzer. As the particles move through a strong electromagnetic field, they are separated according to their electrophoretic mobility and then enumerated by a particle counter.

The principles of differential ion mobility, and their application to the analysis of biomolecules, have been extensively reviewed elsewhere (Guha S, et al., Trends Biotechnol. 2012; 30:291-300; Kaddis C S, Loo J A., Anal Chem. 2007; 79:1778-84; Flagan R C., KONA Powder Part J. 2008; 254-8). Briefly, aqueous HDL particles (or other analytes in solution) are first converted to highly charged, gas-phase ions by electrospray ionization.

Following ESI, ions pass near a²¹⁰Po α-source, where most are neutralized by ionized air (FIG. 15A). The remaining charged species assume a Fuchs charge distribution, which allows the proportion of singly charged cations to be calculated (Fuchs N A, Geofis Pura E Appl. 1963; 56:185-93). Polydisperse ions then enter the differential ion mobility analyzer, where they quickly assume the velocity of the air moving in the y-direction (FIG. 15B). In the differential mobility analyzer, only singly charged cations are separated according to their electrophoretic mobilities. A particular ion's velocity perpendicular to the laminar airflow is dependent on the force exerted by an electromagnetic field (F_{E}) and the counteracting drag force (F_{drag}). Importantly, drag force is a function of both particle size and shape. Depending on the voltage applied, only particles of a certain electrophoretic mobility successfully traverse the differential mobility analyzer, exit the slit, and enter the condensation particle counter (CPC), where they are detected and quantified. In the CPC (FIG. 15C), particles pass through a chamber of saturated water vapor at 75° C. Condensed water increases the effective diameter of each particle, making it detectable by laser light scattering. Differential mobility analyzer size distribution spectra are generated by scanning the applied voltage while recording the abundance of particles of known electrophoretic mobility. Here, electrophoretic mobilities are expressed as “particle diameter”—corresponding to the calculated diameter of a singly-charged, spherical particle with the same electrophoretic mobility. to account for the fact that total particle concentration is different than that determined by A₂₈₀ due to the presence of multiple oligomers.

IMA Instrumentation and Operation.

Analyses were performed on a scanning mobility particle sizer spectrometer (TSI Inc., Shoreview, Minn., model 3080N) fitted with a nano-differential mobility analyzer (TSI Inc., model 3085) and a charge-reducing electrospray ionization source (CR-ESI; TSI Inc., model 3480). The CR-ESI unit was coupled with an autosampler. The differential mobility analyzer scanned particles 5 to 30 nm in diameter in 240 s. Typical electrospray settings were: voltage 2 kV, CO₂ flow 0.15 L/min, and air-flow 1.5 L/min. Monodisperse particles exiting the differential mobility analyzer were detected by a condensation particle counter (TSI Inc., model 3788). Samples were introduced into the electrospray chamber every 15 min by automated loop injections. To limit cross-contamination, the system was equilibrated for 10 min after each injection before data acquisition. Sample carryover was <0.5%.

Deconvolution of HDL Spectra.

IMA spectra were expressed in units of aerosol particle concentration per size bin ([number/cm³]/size bin) with an algorithm supplied by the instrument's manufacturer (Aerosol Instrument Manager, v9.0.0.0, TSI Inc.) (Hoppel W A, J Aerosol Sci. 1978; 9:41-54). Size distribution spectra of human HDL were then analyzed, using open-source curve-fitting software (Fityk version 1.2.0 for Mac (Wojdyr M., J Appl Crystallogr. 2010; 43:1126-8)). Using a custom script, spectra were fitted automatically with 3 Voigt probability distribution curves corresponding to the 3 HDL subspecies. The software iteratively adjusts the peak parameters to minimize the weighted sum of squared residuals, or x². All peak parameters were unfixed but limited in range allowing for adaptive deconvolution of the highly variable HDL size distribution profiles observed in human plasma. Finally, the HDL subspecies' peak areas were converted into aqueous particle concentrations, using glucose oxidase calibration curves.

Calibration Curves of Isolated Proteins.

Solutions of purified proteins were prepared gravimetrically in H₂O. Exact concentrations were determined by absorbance at 280 nm. Solutions were further diluted in NH₄AOc (5 mM, pH 9.2) prior to IMA. Typically, serial dilutions of glucose oxidase (10-1.25 μg/mL) were used for calibration. Particle concentrations of individual protein oligomers were calculated to account for the fact that total particle concentration was different than that determined by A₂₈₀ due to the presence of multiple oligomers.

Calculating Particle Concentration from Protein Concentration and Oligomer Distribution.

Protein concentrations, in ug/mL, were determined by A₂₈₀. These units were converted to molar concentrations using their monomeric molecular weights. To calculate the concentration each oligomer observed in the IMA spectra, the following formula was applied:

$${O}_{x} = {P}_{tot} [ \frac{{A}_{x}}{{\sum }_{n = 1}^{i} n \cdot {A}_{n}} ] ,$$

where Oₓ is the molar concentration of the oligomer x, Pₜₒₜ is the molar concentration of the monomer (calculated from A₂₈₀), Aₓ is the peak area of oligomer x, A, is the peak area of the n^{th} oligomer, n is the order of the n^{th} oligomer, and i is the highest order oligomer observed. This formula accounts for the fact that higher order oligomers have more mass per particle. For the clinical samples, the limits of quantitation shall be bounded by the range of the standard curve constructed as described above. None of the HDL samples analyzed here had peak areas outside these limits.

Analysis of Reconstituted HDL.

Discoidal reconstituted HDL (rHDL) was prepared as previously described (Cavigiolio G, et al., Biochemistry (Mosc). 2008; 47:4770-9). The protein concentration of the rHDL particles (9.6 nm hydrated diameter) was determined by modified Lowry assay (Thermo #23240). Serial dilutions were prepared (5 mM NH₄OAc, pH 9.2) and quantified by calibrated IMA. To validate calibrated IMA, duplicate analyses of two independent rHDL preparations were performed.

Analysis of Gold Nanoparticles.

Stock solutions of gold nanoparticles (10 nm; NanoXact from nanoComposix) were concentrated by centrifugation using the manufacturer's recommended protocol. Particle concentration of the final solution was determined by absorbance at 521 nm. Serial dilutions were then prepared (5 mM NH₄OAc, pH 9.2) and quantified by calibrated IMA. To validate calibrated IMA, duplicate analyses of two independent gold nanoparticle preparations were performed.

Clinical Population.

All subjects provided signed informed consent, and all protocols were approved by the University of Washington Institutional Review Board (IRB #32967B). Forty blood samples were randomly selected from those of 375 subjects with severe carotid cerebrovascular disease enrolled in the CLEAR study (Jarvik G P, et al., Arterioscler Thromb Vasc Biol. 2000; 20:2441-7). Forty samples were also selected from those of the study's >1000 controls. Subjects were matched by sex and diabetic status.

Sample size was determined by power calculations based on preliminary HDL-P data. Selection criteria were: age 55 to 80 years, HDL-C 30 to 80 mg/dL, triglycerides <300 mg/dL. All baseline characteristics of study subjects, except HDL-P, were determined by CLEAR Study investigators and clinical laboratories. CCVD and control subjects were matched by sex and diabetic status. All subjects were on statin therapy. All CCVD subjects had carotid MRI or angiography at a Seattle-area hospital. Subjects with >80% carotid stenosis unilaterally or bilaterally or who had undergone a carotid endarterectomy were considered cases. Control subjects were recruited using clinical databases that excluded anyone with atherosclerosis-related diagnoses. These subjects then underwent a carotid ultrasound. Subjects with <15% carotid stenosis bilaterally were kept as controls.

Statistical Analyses.

Statistical tests were performed using R (v2.15.1) or Prism (v4.0; Graphpad). All t-tests were two-tailed and uncorrected. Correlations were evaluated using the method of Pearson. Odds ratios and their confidence intervals were extracted from generalized linear models constructed in R. For all analyses, P values <0.05 were considered significant.

Calibrated IMA Precision:

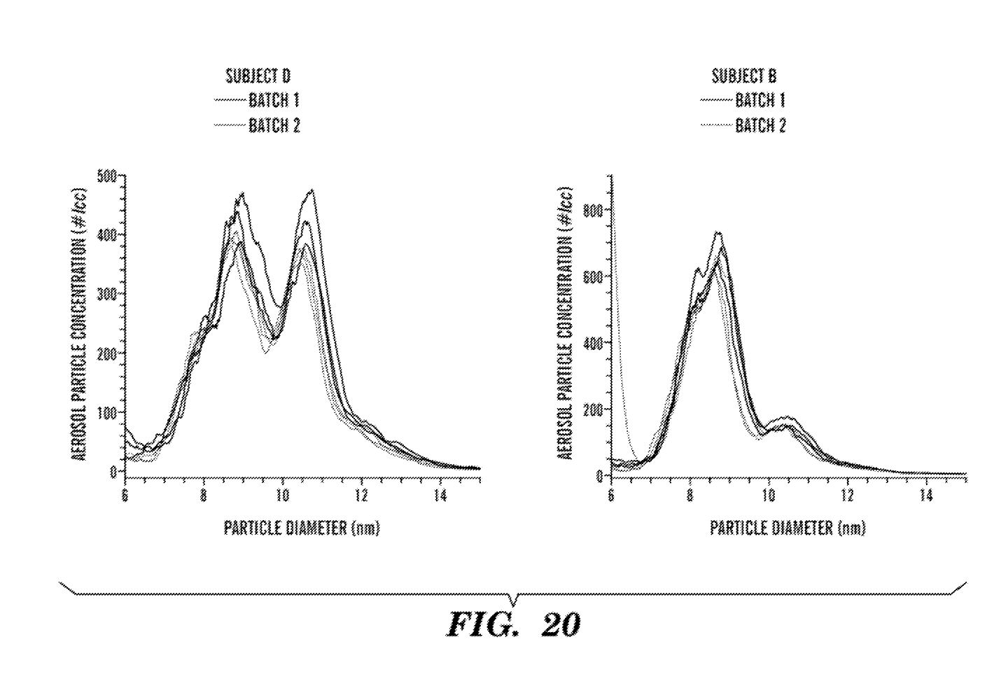

Analytical (or technical) Variability. See FIG. 20. Single isolated HDL preparation was injected and analyzed by IMA 6 times during 18 h (the total analysis time for an entire plate of 72 HDL samples). Each spectrum was processed and deconvoluted in the manner used for the clinical samples. These experiments served two purposes: 1) they established the analytical variability (or technical variability) of calibrated IMA and spectral deconvolution, 2) they demonstrate that HDL samples are stable in the IMA buffer over the time of analysis. The analytical coefficient of variability (CV) was 5.8% for total HDL-P.

Calibrated IMA Precision: Inter-Assay Variability.

HDLs from 12 plasma samples were isolated and analyzed in triplicate by calibrated IMA. All samples were analyzed in the same manner as the clinical samples. Triplicate isolations and analyses of individual samples were performed in parallel, and the same standard curve was used to calibrate replicates. For total HDL particle concentration, the mean inter-assay CV was 6.2%.

Calibrated IMA Precision: Intra-Assay Variability.

HDLs from 12 plasma samples were independently isolated and analyzed by calibrated IMA three separate times. All analyses were performed in the same manner as those of the clinical samples. Independent isolations and analyses took place on different days; a unique calibration curve (GOx) was produced for each batch. For total HDL particle concentration, the mean intra-assay CV was 11.4%.

Calibrated IMA Robustness: Freeze-Thaw Effects.

See FIGS. 13A-13B. Clinical samples are often received as plasma that has been frozen and stored at −80° C. In certain instances, however, frozen plasma samples may be thawed and refrozen more than once. To determine if freeze-thaw cycles affect HDL particle concentrations, aliquots of plasma from four individuals were subjected to one, two, or three rounds of freezing and thawing, and subsequently determined HDL particle concentrations and size by calibrated IMA. Each analysis was performed in triplicate. Particle concentration did not change significantly after one, two, or three freeze/thaw cycles. This stability applied to all three HDL subspecies as well as to total HDL-P. In three plasma samples, the sizes of the HDL subspecies also remained stable. In one plasma sample, the average sm-HDL particle size shifted slightly (0.11 nm) after three freeze/thaw cycles.

Calibrated IMA Robustness: Particles Prepared in Different Laboratories.

rHDL particles were prepared in an independent laboratory and shipped on ice to a different laboratory for analysis. Particle concentrations of rHDL determined by total protein (30.6 nM) and in triplicate by calibrated IMA (26.1 nM) differed by <15%.

Calibrated IMA Robustness: Anti-Coagulant Effects.

Two blood samples were collected in immediate succession from each of 4 study subjects. One set was anticoagulated with EDTA and the other with heparin. Triplicate analyses showed that the type of anticoagulant used had no significant effect on particle concentration for any of the three HDL subspecies or total HDL. Additionally, no differences in HDL subspecies size were observed.

Apparent Molecular Weights by IMA.

The relationship between particle diameter determined by IMA and molecular weight (MW) has been extensively studied (Guha S, et al., Trends Biotechnol. 2012; 30:291-300; Kaddis C S, Loo J A., Anal Chem. 2007; 79:1778-84; Kapellios E A, et al., Anal Bioanal Chem. 2011; 399:2421-33; Kaddis C S, et al., J Am Soc Mass Spectrom. 2007; 18:1206-16; Bacher G, et al., J Mass Spectrom JMS. 2001; 36:1038-52). The correlation is robust, though it can vary slightly between instruments. Therefore, the observed diameters of reference proteins were plotted against their molecular weights. Each protein was measured independently at least 12 times. A power-series function (y=−0.0043x^{0.9177}+1.377x^{0.3727}) best fit the data (r²=0.9987), as in previous reports of similar analyses (Bacher G, et al., J Mass Spectrom JMS. 2001; 36:1038-52). Using this curve, the apparent MW of reconstituted HDL was 174,000 Da, in close agreement with MWs determined by other methods (Marty M T, et al., Anal Chem. 2012; 84:8957-60; Bayburt T H, Sligar S G, FEBS Lett. 2010; 584:1721-7; Cavigiolio G, et al., Biochemistry (Mosc). 2008; 47:4770-9), suggesting that IMA is a relatively accurate method for determining MW.

### Results

Calibrated IMA Quantifies Proteins with Different Molecular Weights (MWs) and Isoelectric Points (pIs).

A key assumption of calibrated IMA is that different particles elicit similar responses when analyzed by the same instrument. To test this assumption, the linearity of the ion mobility signal response was first explored by analyzing serial dilutions of highly purified glucose oxidase (MW_{dimer} 160,000; pI, 4.2) (FIG. 1A). IMA spectral peak areas of glucose oxidase (monomers and dimers) were plotted against particle concentrations calculated from the total protein concentration determined by A₂₈₀ (FIG. 1B). Linear (r²>0.99) concentration-dependent responses were observed for the dimer, the monomer, and total particle concentration. Calibration curves routinely had r² values >0.99.

To determine how particle size and physiochemical properties (e.g., pI) affect instrument response, two additional proteins were interrogated in the same manner. IMA of serial dilutions of catalase (MWₜₑₜᵣₐₘₑᵣ, 240,000; pI, 5.6) and transferrin (MWₘₒₙₒₘₑᵣ 80,000; pI, 6.2-6.6) both yielded linear, concentration-dependent responses similar to those obtained with glucose oxidase. Importantly, all three proteins produced calibration curves with essentially equivalent slopes and y-intercepts. Indeed a single regression line, fit to the superimposed data (FIG. 1C), had an r²=0.98 and passed near the origin.

These observations indicated that proteins of different molecular weights, oligomeric distributions, and isoelectric points all produced similar instrument responses. For routine analyses, glucose oxidase was used as the working calibrant due to its convenient particle diameter near the center of the HDL size-distribution and its stability in aqueous solution.

Calibrated IMA Quantifies the Absolute Concentration of Reconstituted HDL and Gold Nanoparticles.

Reconstituted discoidal HDL (9.6 nm diameter) was next used to determine whether calibrated IMA can accurately quantify HDL-P. These particles were selected because they resemble native HDL and contain two apoA-I molecules per particle (Cavigiolio G, et al., Biochemistry (Mosc). 2008; 47:4770-9; Swaney J B., J Biol Chem. 1980; 255:8798-803), allowing one to establish the concentration of stock solutions based on protein content. When particle concentrations determined by calibrated IMA were plotted against concentrations calculated from total protein (FIG. 1D), the data were linear (r²=0.98) and had a slope close to one (0.99). Gold nanoparticles (˜10 nm diameter) were similarly quantified, whose concentration determined by absorbance at 521 nm. Once again, the two orthogonal methods yielded nearly identical results for particle concentration (FIG. 1E). In separate experiments, the concentration of rHDL prepared in another laboratory and shipped for analysis was determined. Particle concentrations determined in triplicate by IMA (26±1 nM) and by total protein (30.4 nM) differed by <15%.

Calibrated IMA Quantifies Total HDL-P and Three Subspecies in Human Plasma.

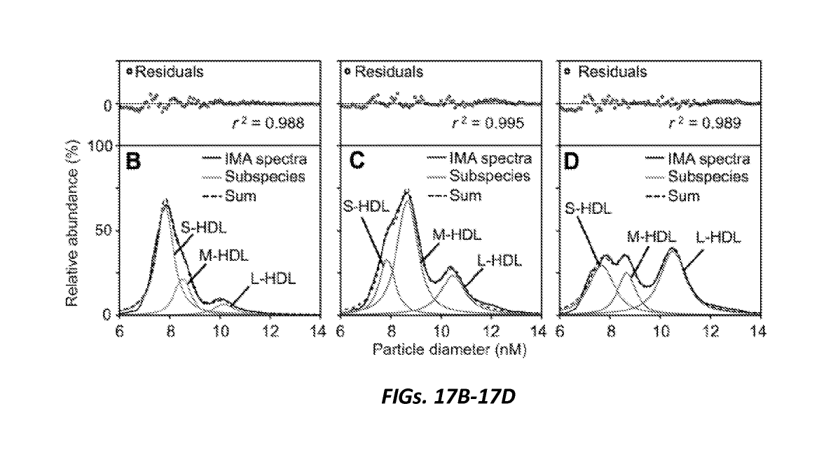

The workflow for determining HDL-P by calibrated IMA is shown in FIG. 17A. To summarize, total lipoproteins were isolated from plasma by a single ultracentrifugation (ρ=1.21 g/mL) step (Havel R J, et al., J Clin Invest. 1955; 34:1345-53) and then dialyzed the preparation to remove salts (which interfere with IMA). After diluting the samples, differential mobility analysis was used to determine the size distribution and uncorrected particle concentration of the isolated HDL species. Because electrophoretic mobility depends chiefly on size, IMA data are expressed in terms of particle diameter, which corresponds to the calculated diameter of a singly charged, spherical particle with the same electrophoretic mobility. For each spectrum, three HDL subspecies (small, medium, large) were deconvoluted by unsupervised, iterative curve-fitting (FIGS. 17B-17D). Finally, HDL peak areas were directly converted to HDL-P, using the calibration curve.

Using this approach, HDL-P in 40 control subjects (<15% carotid intimal thickening) and 40 subjects with severe carotid cerebrovascular disease (CCVD; >80% carotid stenosis by MRI) enrolled in the CLEAR study (Jarvik G P, et al., Arterioscler Thromb Vasc Biol. 2000; 20:2441-7) was determined. The clinical characteristics of the two groups are presented in Table 1. The mean total HDL-P obtained in all 80 subjects by calibrated IMA was 13.4±2.4 μM (mean±SD), with a mean value for plasma apoA-I of 48.8 μM determined by a clinical laboratory.

Calibrated IMA consistently identified 3 major HDL subspecies in plasma from the 80 subjects. They were small HDL (S-HDL, average diameter 7.9 mm), medium HDL (M-HDL, 8.6 mm), and large HDL (L-HDL, 10.4 mm) (Rosenson R S, et al., Clin Chem. 2011; 57:392-410). By first calibrating the IMA instrument with proteins of known MW, the apparent molecular masses of the three subspecies: ˜120 (small), ˜160 (medium), and 270 (large) kDa (FIGS. 14A-14B) can also be determined. These results agree well with direct measurements of HDL's molecular mass by sedimentation ultracentrifugation (Scanu A, Reader W, Edelstein C., Biochim Biophys Acta BBA—Protein Struct. 1968; 160:32-45). Additional HDL subspecies, corresponding to very small HDL (˜100 kDa) and very large HDL (˜500 kDa) (Rosenson R S, et al., Clin Chem. 2011; 57:392-410), appeared too infrequently to be quantified reproducibly. In the current implementation of calibrated IMA, the bounds of the standard curve represent the upper and lower limits of quantitation. No samples showed peak areas outside these values for any HDL subspecies.

When the same HDL preparation was repeatedly analyzed (n=6), the total HDL-P coefficient of variation (CV) was <6% and the proportion of subspecies was consistent (CVs <10%). When plasma samples (n=12) were subjected to multiple independent isolations and analyses (n=3), intra-assay CV was <7% and inter-assay CV was <12% (Table 2, FIG. 20).

The distribution of subspecies in the HDLs of the 80 subjects differed strikingly. While certain samples were composed almost entirely of S-HDL, others were mostly L-HDL, though the majority fell between these extremes. The mean composition was 42% small, 44% medium, and 14% large HDL. While the relative abundance of HDL subspecies varied dramatically, the diameters of the subspecies particles were remarkably consistent for all subjects (size CVs were <3%). A correlation matrix of HDL-P and lipid values is tabulated in Table 3.

Subspecies Distributions Explain Discordant Values for HDL-P and HDL-C.

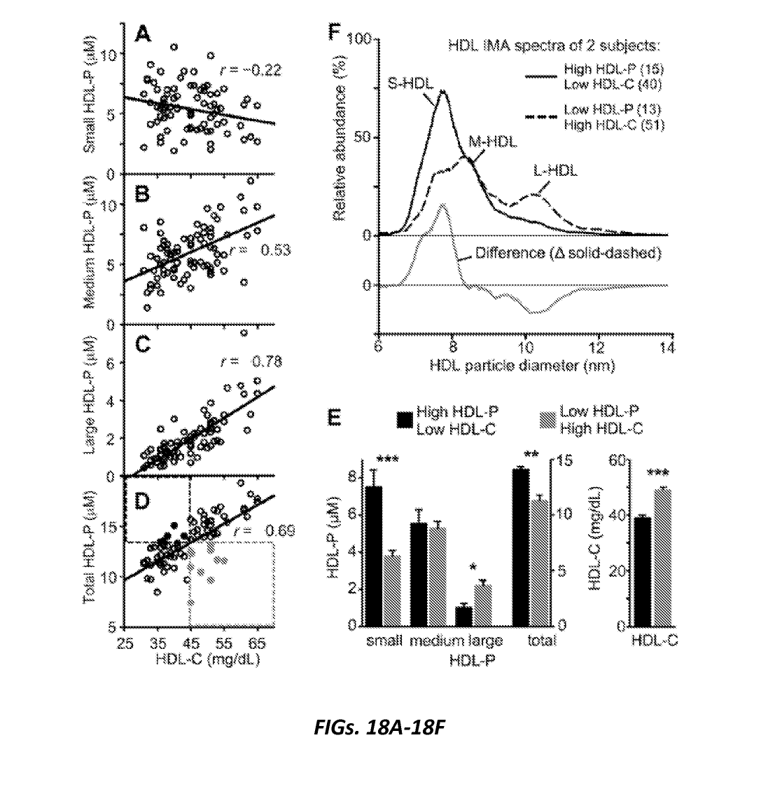

The relationship between HDL-P and HDL-C in all 80 subjects was next determined (FIGS. 18A-18D). The concentration of HDL-C was determined on plasma by a clinical laboratory. HDL-C predicted >60% of the variance in L-HDL-P (r=0.78, P<0.0001), whereas it predicted <30% of the variance in M-HDL-P (r=0.53, P<0.0001). The concentration of S-HDL did not correlate with HDL-C but trended inversely (r=−0.22). Total HDL-P correlation with HDL-C was moderate (r=0.69, P<0.0001). The relationships between HDL-P (total and subspecies) and plasma apoA-I were similar to the HDL-C correlations described above (Table 3). There was little correlation of HDL-P with concentration of LDL cholesterol or other lipids (Table 3).

HDL-C explained only ˜50% of the variation in total HDL-P (FIG. 18D). Consistent with this observation, certain subjects showed discordant values of HDL-P and HDL-C. The variable cholesterol content of individual HDL particles (Shen B W, et al., Proc Natl Acad Sci. 1977; 74:837-41; Huang R, et al., Nat Struct Mol Biol. 2011; 18:416-22) suggested that subspecies' distributions might explain the two metrics' conflicting values. The subset of subjects (n=5) with both high HDL-P (>mean) and low HDL-C (<mean) was therefore compared with those (n=10) who had both low HDL-P (<mean) and high HDL-C (>mean) (FIGS. 18D, 18E). The latter had twice the concentration of L-HDL particles (2.2 vs. 1.0 μM; P=0.02). Conversely, the subjects with high HDL-P/low HDL-C had nearly twice the concentration of S-HDL particles (7.5 vs. 3.8 μM; P=0.0003). Although the two groups had markedly different HDL-C (P=0.0002), they had similar concentrations of M-HDL particles. Calibrated IMA spectra of representative subjects from each group are shown in FIG. 18F.

HDL-P Associates with Carotid Cerebrovascular Disease Independently of HDL-C.

To explore whether calibrated IMA can be a clinically useful alternative to HDL-C measurements, HDL-P in control subjects (n=40) was compared with subjects with severe carotid cerebrovascular disease (CCVD; n=40), a major risk factor for stroke. The subjects' characteristics are summarized in Table 1.

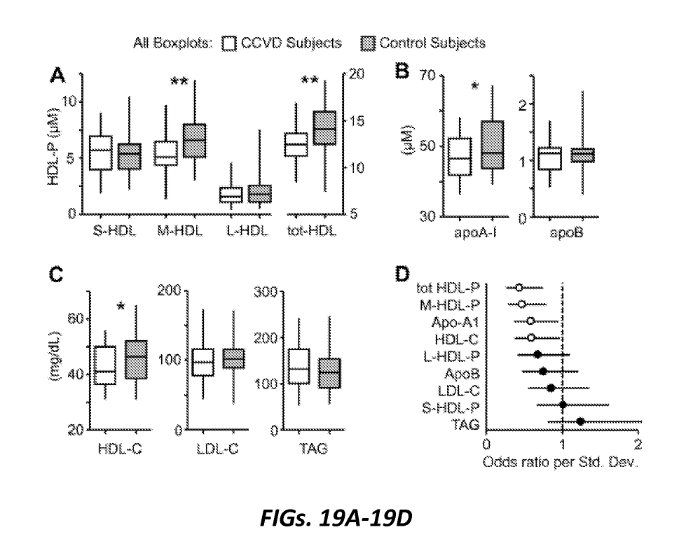

Compared with the controls, the subjects with carotid disease had significantly lower levels of HDL-C, apoA-I, M-HDL-P, and total HDL-P (P=0.04, 0.03, 0.004 and 0.002, respectively) (FIGS. 19A-19C). Unadjusted odds ratios (FIG. 19D) revealed that total HDL-P and M-HDL-P were the strongest predictors of CCVD, followed by HDL-C and apoA-I; no other traditional lipid risk factors quantified by a clinical laboratory were significant predictors in this population.

Importantly, differences in total HDL-P and M-HDL-P remained significant after adjustment for HDL-C (P=0.02 and 0.04, respectively). After adjustment for LDL and triglycerides, HDL-C no longer differed significantly between groups (P=0.06), while both M-HDL and total HDL-P remained strong predictors of CCVD (P=0.003 and 0.009, respectively). Adding age and sex to this model did not affect the significance of HDL-P. Collectively, these observations indicate that HDL-P can provide clinical information about CVD risk that is independent of other traditional lipid risk factors.

### DISCUSSION

The concentration and size of HDL particles in plasma, HDL-P, can represent a metric that more accurately assesses CVD risk than HDL-C.

IMA of proteins of different sizes and physiochemical properties yielded linear calibration curves that were essentially superimposable, suggesting that protein standards could be used to quantify other particles of unknown concentration. Consistent with this proposal, the concentrations of reconstituted HDL particles and gold nanoparticles determined by calibrated IMA were in excellent agreement with concentrations determined by orthogonal methods. Taken together, these observations strongly suggest that calibrated IMA can quantify particles in aqueous solution that range widely in size and composition.

Calibrated IMA was next used to investigate the size and concentration of HDL particles in human plasma. The three subspecies closely matched the sizes of HDL particles defined by ultracentrifugal Schlieren patterns and non-denaturing 2D gradient gel electrophoresis (Rosenson R S, et al., Clin Chem. 2011; 57:392-410; Delalla O F, et al., Am J Physiol. 1954; 179:333-7; Asztalos B F, et al., Biochim Biophys Acta. 1993; 1169:291-300). Thus, S-HDL, M-HDL, and L-HDL likely correspond to α3/4-, α2-, and α1-HDL, respectively. In contrast, non-calibrated IMA detected only two subspecies: large HDL and small HDL (Caulfield M P, et al., Clin Chem. 2008; 54:1307-16). The ability to quantify three subpopulations of HDL likely reflects differences in the methods used to isolate the HDL and the adaptive curve fitting algorithm, which permits deconvolution of partially overlapping HDL subspecies.

A key issue was whether the approach described herein recovered HDL quantitatively from plasma. Immunoblot analysis of material prepared by ultracentrifugation from four individuals indicated that ˜80% of the apoA-I in the HDL fraction was recovered. It is noteworthy that 5-10% of plasma apoA-I is unassociated with lipoproteins (Rye K-A, Barter P J., Arterioscler Thromb Vasc Biol. 2004; 24:421-8). Assuming that 10% of apoA-I is indeed not associated with HDL, it was estimated that the recovery of small, medium and large HDLs—the particles quantified by calibrated IMA—approaches 90%.

A fundamental unresolved issue is the concentration of HDL particles in blood, which, along with subspecies distribution, is likely to impact HDL's functions. In seven independent studies, the mean total HDL-P reported by non-calibrated IMA studies was 5.3 μM, while the average plasma apoA-I concentration was 51 μM (Table 4). These values imply a mean stoichiometry of almost 10 apoA-I molecules per HDL particle. In contrast, HDL particle concentrations derived from NMR analyses were ˜30 μM (Table 4), indicating a stoichiometry of ˜1.6 apoA-I molecules per HDL particle. The mean total HDL-P obtained by calibrated IMA was 13.4 μM with a mean plasma apoA-I value of 48.8 μM, implying 3.6 apoA-I per HDL if all HDL particles contain apoA-I. This stoichiometry is in excellent agreement with abundant biochemical data suggesting a mean of 3-4 apoA-I/HDL and with our current understanding of HDL structure (Shen B W, et al., Proc Natl Acad Sci. 1977; 74:837-41; Huang R, et al., Nat Struct Mol Biol. 2011; 18:416-22). Importantly, this observation further supports the proposal that HDL was recovered in near quantitative yield from plasma.

A striking feature of the clinical data was the marked variability in the abundance of HDL subspecies in different subjects. Among individual subjects, for example, the percentage of M-HDL ranged from <15% to >70%; S-HDL and L-HDL showed similar variation. This HDL heterogeneity highlights the need for a flexible data processing approach.

It is noteworthy that ˜20% of the subjects in the clinical population had high HDL-P levels (>mean) and low HDL-C values (<mean) or low HDL-P (<mean) and high HDL-C (>mean) HDL-C values. These differences in turn reflected major differences in the relative abundance of S-HDL and L-HDL particles. These results support the notion that HDL-P can vary independently from HDL-C and that differences in the proportions of subspecies could account for the discrepancy.

In a clinical population, low total HDL-P associated strongly and inversely with severe carotid cerebrovascular disease. Notably, M-HDL particles were selectively depleted, suggesting that the abundance of a specific HDL subpopulation was reduced in this clinical population. M-HDL only moderately correlated with HDL-C, strongly suggesting that quantifying specific subpopulations of HDL particles might offer information distinct from HDL-C. Importantly, differences in total HDL-P and M-HDL-P remained significant after adjustment for HDL-C, suggesting that HDL-P can offer clinically relevant information beyond HDL-C. The association of low HDL-P with carotid disease persisted after adjustment for other risk factors, including LDL-C, triglycerides, age, and sex.

In conclusion, a method for determining the size and concentration of HDL in human plasma is described herein. The method leverages empiric calibration and was validated by measuring particles of known concentration. Quantifying HDL-P yielded a value for the stoichiometry of apoA-I per HDL particle that fits well with our current understanding of HDL structure. HDL-P was also a strong and independent predictor of CCVD status in a clinical population.

1. A method of characterizing particles in a sample solution, the method comprising:
(i) converting a portion of the particles in the sample solution into gas-phase ions;
(ii) performing an ion mobility measurement on the gas-phase ions, whereby the gas-phase ions are enumerated according to size, thereby producing data relating particle size to relative abundance;
(iii) processing the data by using a calibration regression, wherein the calibration regression is obtained by:
(a) performing steps (i) and (ii) on reference particles of known solution-phase concentration; and
(b) constructing the regression relating total number of enumerated gas-phase ions of the reference particles to the known solution-phase concentration;
and
(iv) quantitatively determining particle concentration in the sample solution based on the processing.

2. The method of claim 1, wherein step (ii) produces a spectrum of particle size distribution.

3. The method of claim 2, further comprising superimposing a plurality of distribution curves over the spectrum, each distribution curve representing a subpopulation of the gas-phase ions according to size, and iteratively adjusting parameters of the distribution curves to minimize the difference between the spectrum and sum of the distribution curves.

4. The method of claim 3, wherein the distribution curve is selected from the group consisting of a Gaussian, a split Gaussian, a Voigt, a split Voigt, a Pearson7, a split Pearson7, a Lorentzian, and a split Lorentzian distribution.

5. The method of claim 1, wherein the ion mobility measurement comprises introducing the gas-phase ions into an electromagnetic field having an effect on the translation of the ions, thereby inducing an electrophoretic motion.

6. The method of claim 1, wherein the conversion into gas-phase ions is done by electrospray ionization.

7. The method of claim 1, wherein the particles and reference particles are each independently selected from the group consisting of biological particles, inorganic particles, metallic particles, metallo-organic particles, organic particles, polymeric particles, and a combination thereof.

8. The method of claim 7, wherein the biological particles are biological cells, proteins or aggregates thereof, or lipoproteins.

9. The method of claim 8, wherein the lipoproteins are selected from the group consisting of whole HDL, fractionated HDL, whole LDL, fractionated LDL, whole VLDL, fractionated VLDL, and a combination thereof.

10. The method of claim 1, wherein the reference particles comprises nanoparticles selected from the group consisting of gold, silver, polystyrene, silica, purified proteins, and a combination thereof.

11. The method of claim 10, wherein the purified protein is glucose oxidase.

12. The method of claim 1, wherein the sample solution is an aqueous solution.

13. The method of claim 12, wherein the aqueous solution is a biological sample.

14. The method of claim 13, wherein the biological sample is selected from the group consisting of blood, plasma, serum, urine, cerebrospinal fluid, and saliva.

15. The method of claim 12, further comprising dialyzing the aqueous solution to substantially remove salts.

16. The method of claim 1, wherein the reference particles are of known molecular weight.

17. The method of claim 16, further comprising determining the molecular weight of the particles being characterized.

18. The method of claim 1, wherein the reference particles are of known size.

19. A method of determining if a subject is at risk to develop or is suffering from a cardiovascular disease, the method comprising: measuring, in a biological sample obtained from the subject, the size and concentration of HDL particles according to the method of claim 1.

20. The method of claim 19, wherein the HDL particles are selected from the group consisting of very small HDL particles, small HDL particles, medium HDL particles, large HDL particles, very large HDL particles, and a combination thereof

21. The method of claim 19 or 20, further comprising measuring lipoproteins other than HDL.

22. The method of claim 19, wherein the cardiovascular disease is selected from the group consisting of atherosclerosis, coronary vascular disease, ischemic heart disease, myocardial infarction, angina pectoris, peripheral vascular disease, cerebrovascular disease, endothelial dysfunction, and stroke.

23. The method of claim 19, wherein the biological sample is selected from the group consisting of blood, plasma, and serum.

24. The method of claim 19, wherein the subject is a mammal.

25. The method of claim 24, wherein the mammal is a human.

26. A method of determining if a subject has lecithin-cholesterol acyltransferase deficiency (LCAT), the method comprising:
(i) measuring, in a biological sample obtained from the subject, the concentration of HDL particles; and
(ii) determining that the subject has LCAT if the concentration of very small HDL particles is at or above a first reference level, and the concentration of at least one other subpopulation of HDL particles is below a second reference level.

27-36. (canceled)

37. A method of determining if a subject is at risk to develop or is suffering from atherosclerosis, the method comprising:
(i) measuring, in a biological sample obtained from the subject, the concentration of HDL particles; and
(ii) determining that the subject is at risk to develop or is suffering from atherosclerosis if the concentration of HDL particles is below a reference level.

38-49. (canceled)

50. A method of determining if a subject is at risk to develop or is suffering from endothelial dysfunction, the method comprising:
(i) measuring, in a biological sample obtained from the subject, the concentration of HDL particles; and
(ii) determining that the subject is at risk to develop or is suffering from endothelial dysfunction if the concentration of HDL particles is below a reference level.

51-59. (canceled)
